(19) United States
(12) Patent Application Publication
(10) Pub. No.: US 2026/0035726 A1
(43) Pub. Date: Feb. 5, 2026

(54) RECOMBINANT POLYADENYLATION SIGNAL SEQUENCES AND USE THEREOF

(71) Applicant: Hoffmann-La Roche Inc., Little Falls, NJ (US)

(72) Inventors: Viktor Mikael HAELLMAN, Basel (CH); Filip ROUDNICKY, Zürich (CH)

(73) Assignee: Hoffmann-La Roche Inc., Little Falls, NJ (US)

(21) Appl. No.: 19/355,204

(22) Filed: Oct. 10, 2025

Related U.S. Application Data
(63) Continuation of application No. PCT/EP2024/059762, filed on Nov. 4, 2024.

(30) Foreign Application Priority Data
Apr. 13, 2023 (EP) 23167813.7

(51) Int. Cl. C12P 21/00; C12N 15/85

(52) U.S. Cl. CPC C12P 21/00; C12N 15/85; C12N 2800/107; C12N 2830/50

(57) ABSTRACT

The present invention relates to improved recombinant polyadenylation signal sequences and use thereof.

### CROSS REFERENCE TO RELATED APPLICATIONS

This application is a continuation of International Application No. PCT/EP2024/059762, filed Apr. 11, 2024, which claims priority to EP application Ser. No. 23167813.7 filed Apr. 13, 2023, each of which is incorporated herein by reference in its entirety.

### SEQUENCE LISTING

This application contains a Sequence Listing which has been submitted electronically in XML format and is hereby incorporated by reference in its entirety. Said XML copy, created on Oct. 9, 2025, is named “P38269-US-1-Sequence_Listing.xml” and is 19,149 bytes in size.

### FIELD OF THE INVENTION

The present invention relates to improved recombinant polyadenylation signal sequences and use thereof.

### BACKGROUND OF THE INVENTION

Generating stable, high-level recombinant protein-expressing cell lines is essential for cell engineering applications in research, disease modelling, drug discovery, therapeutics gene expression, and biopharmaceutical production. Achieving robust recombinant protein expression in a desired host cell hinges optimization of the expression vector using various enhancers, promoters, intron, polyA, and regulatory sequences. Various approaches for developing and optimizing the genetic elements used in expression vectors to achieve the desired level of transgene expression have been reported, including assembling blocks of natural or de novo-designed functional elements (Cao et al., 2021; McFarland et al., 2006; Patel et al., 2021; Schlabach et al., 2010), however, with little consideration to creating higher-level multigene expression vectors to coexpress recombinant genes predictably from the same vector. Since most efforts to optimize expression vectors have focused on identifying the optimal sequence for a specific recombinant protein and vector, the translatability of these genetic elements to other applications can be limiting. Moreover, taking one optimized genetic element and replicating it for multiple expression units on a multigene expression vector is often less than ideal, as introducing extended regions of repeat sequences through the replication of regulatory sequence or by reusing identical genetic elements introduces the risk for recombination events to occur during replication of the vector and integration in a target host cell (Bzymek et al., 2001; Finn et al., 1989). In mammalian cells, transcriptional termination and polyadenylation is vital to efficient protein expression of endogenous genes, as well as recombinant genes expressed from an engineered vector. Specifically, the polyA tail added to protein-coding transcripts aid in nuclear export and translation and stability of mRNA by protecting the transcript from enzymatic degradation in the cytoplasm. Some of the more commonly used polyadenylation signal sequences used in vector development include the sequences from the bovine growth hormone (BGH) (Goodwin et al., 1992), human growth hormone (hGH) (Pfarr et al., 1986), simian virus 40 (SV40) (Hans et al., 2000), and rabbit beta-globin (RbG) (Lanoix et al., 1988). However, due to significant differences in size and sequence composition, the choice of polyadenylation signal sequences can significantly impact the characteristics of the expression vector. Larger size will contribute to reduced transfection and integration efficiency during engineering cells and, in the case of viral vector engineering, may increase the size of the genetic cargo beyond the packaging capacity of the viral vector. Differences in sequence composition and functional elements within the polyadenylation signal sequences can lead to heterogeneous expression levels from their respective usage and variability between expression levels in different host cells, as in the case of SV40 and RbG, which are proposed to be more efficient polyadenylation signal sequences compared to others due the presence of additional upstream and downstream functional elements that contribute to termination and polyadenylation (Gil et al., 1987; Schek et al., 1992). Moreover, recent data suggest that even minor variations in sequence composition may contribute to differences in the transcription termination processes and protein expression within the same host cells and between various host cells (Omelina et al., 2022). Hence, there remains a need for improved polyadenylation signal sequences.

### SUMMARY OF THE INVENTION

Herein provided is a repertoire of robust transcriptional termination and polyadenylation (polyA) signal sequences for advanced vector development. The present inventors generated a set of rationally designed recombinant polyadenylation signal sequences based on the minimal core sequence of the RbG polyadenylation signal sequence (Levitt et al., 1989). These recombinant polyadenylation signal sequences are particularly useful for generating multigene expression vectors for engineering mammalian cells due to their small size and defined functional element composition. Moreover, the sequence composition has been specifically designed to accommodate Gibson DNA assembly cloning to facilitate the generation of multigene expression vectors and with a low identical sequence composition (sequence identity) to reduce the risk for DNA recombination events. The resulting recombinant polyadenylation signal sequences of the present invention support notably higher expression levels than the known short polyadenylation signal sequences such as the core RbG polyA sequence (see for example FIG. 5) and levels comparable to larger commonly used polyadenylation signal sequences (see for example FIG. 6). Further, the inventors showed that the recombinant polyadenylation signal sequences of the present invention are minimally affected by the upstream 3'UTR sequence composition (see for example FIG. 7) and can support robust expression in combination with different strength constitutive promoters (see for example FIG. 8). Taken together, the present inventors provided a new, improved set of recombinant polyadenylation signal sequences that can facilitate the development of advanced multigene expression vectors and cell models for research, disease modeling, drug discovery, therapeutics gene expression, and biopharmaceutical production. Further advantageous effects in the context of specific exemplary uses are described herein below.

In one embodiment, provided is recombinant transcriptional unit comprising a nucleotide sequence encoding a polypeptide wherein the nucleotide sequence is operably linked to a recombinant polyadenylation signal sequence, wherein the recombinant polyadenylation signal sequence has a sequence length of less than 100 nucleotides, and wherein a eukaryotic cell transformed with a recombinant nucleic acid comprising the recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity.

In some embodiments, the recombinant polyadenylation signal sequence comprises or consists of a nucleotide sequence having at least 90%, 91%, 92%, 93%, 94%; 95%, 96%, 97%, 98%, 99% or 100% sequence identity to a nucleotide sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

In some embodiments, provided is a recombinant transcriptional unit comprising a nucleotide sequence encoding a polypeptide wherein the nucleotide sequence is operably linked to a recombinant polyadenylation signal sequence, wherein the recombinant polyadenylation signal sequence comprises or consists of a nucleotide sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

In some embodiments, the recombinant polyadenylation signal sequence comprises or consists of a nucleotide sequence selected from the group consisting of SEQ ID NO:6, SEQ ID NO:9, and SEQ ID NO:12.

In one embodiment, provided is recombinant nucleic acid comprising:
(a) a first recombinant transcriptional unit comprising a first nucleotide sequence encoding a first polypeptide operably linked to a first recombinant polyadenylation signal sequence, and
(b) a second recombinant transcriptional unit comprising a second nucleotide sequence encoding a second polypeptide operably linked to a second recombinant polyadenylation signal sequence, wherein the first and second recombinant polyadenylation signal sequence have less than 70%, 65%, 60%, 55%, 50%, 45% or 40% sequence identity.

In some embodiments, the recombinant nucleic acid further comprises:
(c) a third recombinant transcriptional unit comprising a third nucleotide sequence encoding a third polypeptide operably linked to a third recombinant polyadenylation signal sequence, wherein the first and second recombinant polyadenylation signal sequence individually have less than 70%, 65%, 60%, 55%, 50%, 45% or 40% sequence identity with the third recombinant polyadenylation signal sequence.

In some embodiments, the first, second, and where present third recombinant polyadenylation signal sequence have a sequence length of less than 100 nucleotides.

In some embodiments, the first, second, and where present third recombinant polyadenylation signal sequence cannot engage in DNA strand exchange to form a recombination intermediate.

In some embodiments, recombination events between nucleic acid comprising the first recombinant polyadenylation signal sequence and nucleic acid comprising the second recombinant polyadenylation signal sequence are reduced or prevented.

In some embodiments, recombination events between nucleic acid comprising the first recombinant polyadenylation signal sequence and nucleic acid comprising the third recombinant polyadenylation signal sequence are reduced or prevented, and/or wherein recombination events between nucleic acid comprising the second recombinant polyadenylation signal sequence and nucleic acid comprising the third recombinant polyadenylation signal sequence is reduced or prevented.

In some embodiments, the first, second, and where present third polypeptides are expressed in an eukaryotic cell.

In some embodiments, the first recombinant transcriptional unit is a recombinant transcriptional unit as described herein above, and wherein the second recombinant transcriptional unit is a recombinant transcriptional unit as described herein above, and wherein where present the third recombinant transcriptional unit is a recombinant transcriptional unit as described herein above.

In some embodiments, provide is recombinant nucleic acid as described herein above, wherein
(a) the first recombinant transcriptional unit further comprises a first promoter operably linked to the nucleotide sequence encoding the first polypeptide, and
(b) the second recombinant transcriptional unit further comprises a second promoter operably linked to the nucleotide sequence encoding the second polypeptide,
   wherein the first and second promoter have less than 70%, 65%, 60%, 55%, 50%, 45% or 40% sequence identity.

In some embodiments, provide is recombinant nucleic acid as described herein above, further comprising:
(c) where present the first recombinant transcriptional unit further comprises a first promoter operably linked to the nucleotide sequence encoding the first polypeptide,
wherein the first and second promoter have less than 80%, 79%, 78%, 77%, 76%, 75%, 74%, 73%, 72%, 71%, 70%, 65%, or 60% sequence identity with the third promoter.

In some embodiments, provide is recombinant nucleic acid as described herein above, wherein
(i) the first, second, and where present third promoters are active in eukaryotic cells,
(ii) the first promoter drives expression of the first polypeptide,
(iii) the second promoter drives expression of the second polypeptide,
(iv) the third promoter drives expression of the third polypeptide, and/or
(v) the thirst, second, and where present third promoter drive expression of the first, second, and where present third polypeptide, respectively.

In some embodiments, provide is recombinant nucleic acid as described herein above, wherein the first, second, and where present third promoter are individually selected from the group consisting of the hPGK1 promoter, the CMV promoter, and the hEF1α promoter.

In some embodiments, the recombinant nucleic acid comprises at least one vector.

In some embodiments, the recombinant nucleic acid comprises a first vector comprising the first recombinant transcriptional unit, and a second vector comprising the second recombinant transcriptional unit, and where a third recombinant transcriptional unit is present a third vector comprising the third recombinant transcriptional unit.

In some embodiments, the at least one vector comprises a selectable marker operably linked to the first, second, or where present third recombinant transcriptional unit, respectively.

In some embodiments, the selectable marker is selected from the group consisting of the hygromycin selectable marker, the neomycin selectable marker, the G418 selectable markers, dihydrofolate reductase (DHFR), thymidine kinase, glutamine synthetase, asparagine synthetase, tryptophan synthetase, histidinol dehydrogenase, and nucleic acids conferring resistance to puromycin, bleomycin, phleomycin, chloramphenicol, Zeocin, and mycophenolic acid.

In some embodiments, the first, second, and/or where present the third vector comprise a bacterial origin of replication, in particular the pUC19 origin of replication.

In some embodiments, provided is a host cell comprising the recombinant transcriptional unit as described herein above and/or the recombinant nucleic acid as described herein above.

In some embodiments, the host cell is an eukaryotic host cell.

In some embodiments, the host cell is selected from the group consisting of CHO, BHK, HEK, and Sp2/0.

In some embodiments, provided is a recombinant viral vector comprising a vector genome, wherein the vector genome comprises in 5′ to 3′ order:
(i) a 5′ ITR sequence,
(ii) a promoter sequence,
(iii) a sequence encoding a polypeptide,
(iv) a recombinant polyadenylation signal sequence selected from the group consisting of

SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, and
(v) a 3′ ITR sequence.

In some embodiments, the recombinant polyadenylation signal sequence is selected from the group consisting of SEQ ID NO:6, SEQ ID NO:9, and SEQ ID NO:12.

In some embodiments, the recombinant viral vector is selected from the group consisting of a retroviral vector, an adenoviral vector, a helper-dependent adenoviral vector, a hybrid adenoviral vector, a herpes simplex virus vector, a lentiviral vector, a poxvirus vector, an Epstein-Barr virus vector, a vaccinia virus vector, a human cytomegalovirus vectors, a lentiviral vector, an adenoviral vector or an adeno-associated virus (AAV) vector, or a recombinant variant derived therefrom.

In some embodiments, the recombinant viral vector is a recombinant adeno-associated virus (rAAV) vector.

In some embodiments, the AAV capsid is selected from the group consisting AAV1, AAV2, AAV3, AAV4, AAV5, AAV6, AAV7, AAV8, AAV9, AAV 10, AAV11, AAV12, AAV-rh74, AAV-rh10, AAV3B, AAV-2i8 capsid or a variant capsid derived therefrom.

In some embodiments, provided is a method of producing a polypeptide of interest, the method comprising the steps of
(a) providing the host cell as described herein above,
(b) incubating the host cell under conditions suitable for expression of the polypeptide,
(c) recovering the polypeptide of interest from the cell culture.

In some embodiments, provided is a method of producing a polypeptide of interest, the method comprising the steps of
(a) providing a host cell comprising the recombinant nucleic acid as described herein above, wherein the polypeptide of interest is the first polypeptide, and wherein the second and where present third polypeptide are required for or improve the production of the polypeptide of interest,
(b) incubating the host cell under conditions suitable for expression of the first, second, and where present third polypeptide,
(c) recovering the polypeptide of interest from the cell culture, and optionally
(d) formulating the recovered polypeptide of interest for therapeutic use.

In some embodiments, provided is a method of producing a recombinant adeno-associated virus (rAAV) vector, the method comprising the steps of
(a) providing a host cell comprising the recombinant nucleic acid as described herein above, wherein the first polynucleotide sequence encodes for a therapeutic payload, wherein the second nucleotide sequence encodes for viral vector rep and cap protein, wherein the third nucleotide sequence encodes for E4, E2a and VA protein,
(b) incubating the host cell under conditions suitable for production of the recombinant rAAV vector, and
(c) recovering the viral vector from the cell culture, and optionally
(d) formulating the recovered polypeptide of interest for therapeutic use.

In some embodiments, provided is a method as described herein before, wherein the host cell is selected from the group consisting of a CHO cell, a BHK cell, a HEK cell, and a Sp2/0 cell.

In some embodiments, provided is a method as described herein before, wherein the rAAV vector is selected from the group consisting of AAV1, AAV2, AAV3, AAV4, AAV5, AAV6, AAV7, AAV8, AAV9, AAV 10, AAV11, AAV12, AAV-rh74, AAV-rh10, AAV3B, AAV-2i8 vector, or a vector variant derived therefrom.

In some embodiments, provided is the use of a recombinant transcriptional unit for recombinant production of a polypeptide of interest, wherein the recombinant transcriptional unit is as defined herein before.

In some embodiments, provided is the use of a recombinant nucleic acid for recombinant production of a polypeptide of interest, wherein the recombinant nucleic acid is as defined herein above.

### BRIEF DESCRIPTION OF THE FIGURES

FIG. 1 Schematic illustration of the reporter plasmid used to test recombinant polyadenylation signal sequences, consisting of a constitutive promoter (Prom.), enhanced green fluorescent protein (EGFP), P2A self-cleaving peptide sequence, NanoLuc luciferase (Nluc), PEST protein degradation signal, and a 3′ untranslated region (3′UTR), followed downstream by the recombinant polyadenylation signal sequence of interest.

FIG. 2A Schematic illustration of the reporter plasmid with either a BGH or 2× sNRP-1 polyadenylation signal sequences. FIG. 2B Transient testing of the respective reporter constructs in HEK293T, assessed in terms of Nluc expression levels 24 h after transfection. Bars represent the mean ±s.d. of n=16 biologically independent samples normalized to the mean Nluc relative luminescence (RLU) of the BGH polyA encoding reporter plasmid samples (Normalized Luminescence; %).

FIG. 3 Schematic illustration of the herein presented 95 nt recombinant polyadenylation signal sequence design, consisting of (i) a 46 nt heterogenous U-rich upstream sequence element (USE) region designed to contain a unique primer annealing site with a Tm of 70-72° C. compatible with Gibson Assembly, (ii) polyadenylation signal (PAS), (iii) variable spacer region containing two cytosine-adenine (CA) mRNA cleavage sites 15-20 nt downstream of the PAS, (iv) two GU/U-rich downstream sequence element (DSE; DSE1 and DSE2) regions.

FIG. 4A Schematic illustration of the reporter plasmid containing new recombinant polyadenylation signal sequences. FIG. 4B Transient testing of the respective reporter constructs in HEK293T, assessed in terms of Nluc expression levels 24 h after transfection. Bars represent the mean ±s.d. of n=16 independent replicates normalized to the mean Nluc relative luminescence (RLU) of all samples (Normalized Luminescence; %).

FIG. 5A Schematic illustration of the reporter plasmid containing either rabbit beta-globulin polyadenylation signal sequence, as defined by Levitt et al., or one of three selected recombinant polyadenylation signal sequences (polyA-2.3, -3.3, -4.3). FIG. 5B Transient testing of the respective reporter constructs in HEK293T, assessed in terms of Nluc expression levels 24 h after transfection. Bars represent the mean ±s.d. of n=16 independent replicates normalized to the mean Nluc relative luminescence (RLU) of the Levitt et al. polyA encoding reporter plasmid samples (Normalized Luminescence; %).

FIG. 6A Schematic illustration of the reporter plasmid containing either hGH, BGH, SV40, or one of the three selected recombinant polyadenylation signal sequences (polyA-2.3, -3.3, -4.3). FIG. 6B Transient testing of the respective reporter constructs in HEK293T, assessed in terms of Nluc expression levels 24 h after transfection. Bars represent the mean ±s.d. of n=16 independent replicates normalized to the mean Nluc relative luminescence (RLU) of the BGH polyA encoding reporter plasmid samples (Normalized Luminescence; %).

FIG. 7A Schematic illustration of the reporter plasmid containing one of three selected recombinant polyadenylation signal sequences (polyA-2.3, -3.3, -4.3) in combination with three different de novo designed 3′UTR sequences (3′UTR 1, 2, 3). FIG. 7B Transient testing of the respective reporter constructs in HEK293T, assessed in terms of Nluc expression levels 24 h after transfection. Bars represent the mean ±s.d. of n=16 independent replicates normalized to the mean Nluc relative luminescence (RLU) of all samples (Normalized Luminescence; %).

FIG. 8A Schematic illustration of the reporter plasmid containing one of three selected recombinant polyadenylation signal sequences (polyA-2.3, -3.3, -4.3) in combination with three constitutive promoters with different strengths (hPGK1, CMV, hEF1α). FIGS. 8B, 8C and 8D Transient testing of the FIG. 8B hPGK1-, FIG. 8C CMV-, and FIG. 8D hEF1α-driven reporter constructs in HEK293T, respectively, assessed in terms of Nluc expression levels 24 h after transfection. Bars represent the mean ±s.d. of n=16 independent replicates normalized to the mean Nluc relative luminescence (RLU) of all samples tested with the corresponding promoter (Normalized Luminescence; %).

### References

Bzymek et al. (2001). Instability of repetitive DNA sequences: the role of replication in multiple mechanisms. Proceedings of the National Academy of Sciences, 98(15), 8319-8325.

Batt et al. (1995). Characterization of the polyomavirus late polyadenylation signal. Molecular and Cellular Biology, 15:4783-4790

Cao et al. (2021). High-throughput 5′ UTR engineering for enhanced protein production in non-viral gene therapies. Nature communications, 12(1), 4138.

Cole et al. (1985). Identification of sequences in the herpes simplex virus thymidine kinase gene required for efficient processing and polyadenylation. Molecular and Cellular Biology. 5:2104-2113

Finn et al. (1989). Homologous plasmid recombination is elevated in immortally transformed cells. Molecular and Cellular biology, 9(9), 4009-4017.

Gil et al. (1987). Position-dependent sequence elements downstream of AAUAAA are required for efficient rabbit β-globin mRNA 3′ end formation. Cell, 49(3), 399-406.

Gil et al. (1984). A sequence downstream of AAUAAA is required for rabbit β-globin mRNA 3′-end formation. Nature, 312: 473-474

Gimmi et al. (1989). Alterations in the pre-mRNA topology of the bovine growth hormone polyadenylation region decrease poly(A) site efficiency. Nucleic Acid Research, 17(17):6983-98

Goodwin et al. (1992). The 3′-flanking sequence of the bovine growth hormone gene contains novel elements required for efficient and accurate polyadenylation. Journal of Biological Chemistry, 267(23), 16330-16334.

Hans et al. (2000). Functionally significant secondary structure of the simian virus 40 late polyadenylation signal. Molecular and Cellular Biology, 20(8), 2926-2932.

Lanoix et al. (1988). A rabbit beta-globin polyadenylation signal directs efficient termination of transcription of polyomavirus DNA. The EMBO journal, 7(8), 2515-2522.

Levitt et al. (1989). Definition of an efficient synthetic poly (A) site. Genes & Development, 3(7), 1019-1025.

McFarland et al. (2006). Evaluation of a novel short polyadenylation signal as an alternative to the SV40 polyadenylation signal. Plasmid, 56(1), 62-67.

Murthy et al. (1995). The 160-kD subunit of human cleavage-polyadenylation specificity factor coordinates pre-mRNA 3′-end formation. Genes & Development 9:2672-2683

Omelina et al. (2022). Slight Variations in the Sequence Downstream of the Polyadenylation Signal Significantly Increase Transgene Expression in HEK293T and CHO Cells. International Journal of Molecular Sciences, 23(24), 15485

Patel et al. (2021). Control of multigene expression stoichiometry in mammalian cells using synthetic promoters. ACS Synthetic Biology, 10(5), 1155-1165

Pfarr et al. (1986). Differential effects of polyadenylation regions on gene expression in mammalian cells. DNA, 5(2), 115-122

Schek et al. (1992). Definition of the upstream efficiency element of the simian virus 40 late polyadenylation signal by using in vitro analyses. Molecular and Cellular Biology, 12(12), 5386-5393

Schlabach et al. (2010). Synthetic design of strong promoters. Proceedings of the National Academy of Sciences, 107(6), 2538-2543

Takagaki et al. (1997). RNA recognition by the human polyadenylation factor CstF. Molecular and Cellular Biology 17: 3907-3914

Takagaki et al. (1992). The human 64 kDa polyadenylation factor contains a ribonucleoprotein-type RNA binding domain and unusual auxiliary motifs. Proceedings of the National Academy of Sciences 1992; 89:1403-1407

### DETAILED DESCRIPTION OF THE INVENTION

The present inventors have generated improved polyadenylation signal sequences which when integrated into transcriptional units (also called transcriptional cassettes) lead to strong expression of polypeptides of interest. The novel sequences are short which is advantageous for many applications, for example integrating the recombinant polyadenylation signal sequences into transcriptional units which are limited in size (for example in the context of recombinant adeno-associated virus vectors). Furthermore, the present inventors generated multiple recombinant polyadenylation signal sequences that share a low sequence homology, i.e. the sequence identity of the multiple sequences is such as to prevent recombination events. Recombination events may occur in eukaryotic cells if sequences having a high sequence identity come into close proximity to each other, for example if sequences of high sequence identity are integrated into the same genomic locus or in case multiple plasmid vectors sharing sequences of high homology are transfected into a cell.

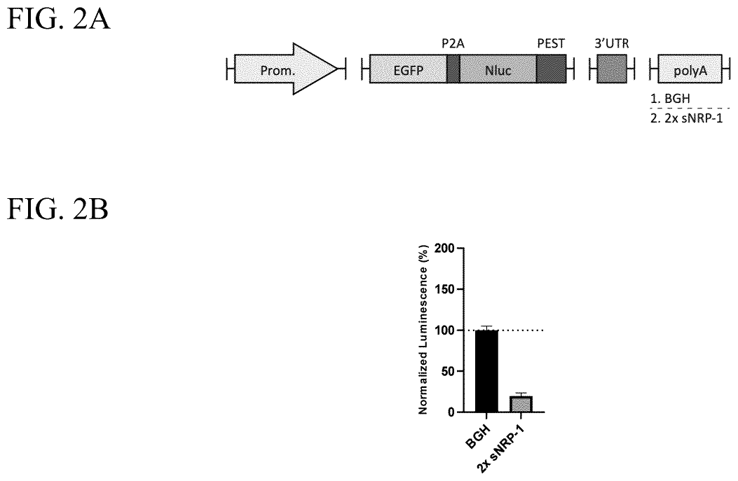

As shown in the Examples, short polyadenylation signal sequences known in the art (such as for example the 2× sNRP-1 signal sequence described in McFarland et al., SEQ ID NO:14, 49 nucleotides long) are not capable of effecting an expression level of a polypeptide of interest comparable to the expression level of longer polyadenylation signal sequences know in the art (such as for example the BGH sequence, SEQ ID NO:16, 208 nucleotides long). This is illustrated for example in FIG. 2B.

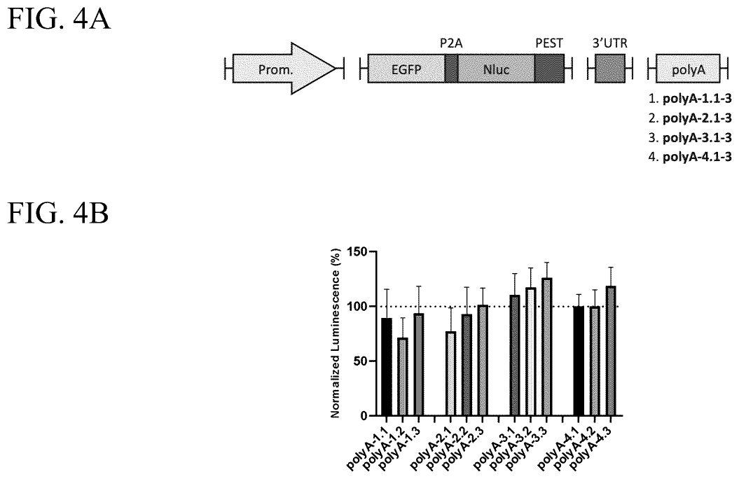

In contrast the new recombinant polyadenylation signal sequences of the present invention (SEQ ID NOs 1-12) effect strong expression levels similar to or higher to as compared to longer polyadenylation signal sequences known in the art (such as for example the hGH, the BGH and the SV40 sequences, SEQ ID NOs:15-17, 122-477 nucleotides long). This is illustrated for example in FIGS. 4B, 5B, and 6B.

Accordingly, herein provided are new and improved recombinant polyadenylation signal sequences. Further provided are recombinant transcriptional units comprising the improved recombinant polyadenylation signal sequences according to the present invention. These transcriptional units can effect strong expression of nucleotide sequences encoding a polypeptide of interest and operably linked to the improved recombinant polyadenylation signal sequences according to the present invention.

In one aspect, provided is recombinant nucleic acid comprising a recombinant transcriptional unit comprising a nucleotide sequence encoding a polypeptide wherein the nucleotide sequence is operably linked to a recombinant polyadenylation signal sequence.

The terms “nucleic acid”, “polynucleotide”, “oligonucleotide” are used interchangeably to mean multiple “nucleotides” (i.e. molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)). As used herein, the terms refer to oligoribonucleotides as well as oligodeoxyribonucleotides. The terms shall also include polynucleosides (i.e. a polynucleotide minus the phosphate) and any other organic base containing polymer. Nucleic acid molecules can be obtained from existing nucleic acid sources (e.g. genomic or cDNA), but may be synthetically produced (e.g. produced by oligonucleotide synthesis).

As referred to herein, a “recombinant” nucleic acid refers to a non-naturally-occurring nucleic acid. A recombinant nucleic acid may also be referred to as a “synthetic” nucleic acid. Similarly, a recombinant transcriptional unit and a recombinant polyadenylation signal sequence refer to non-naturally-occurring transcriptional units and polyadenylation signal sequences, for example comprising or consisting of recombinant nucleic acid. A recombinant nucleic acid may comprise or consist of a polynucleotide sequence that is not comprised and/or encoded by the genome of a naturally-occurring organism (e.g. a wildtype organism). A recombinant nucleic acid may comprise or consist of a polynucleotide sequence which is not comprised in the polynucleotide sequence of a (RNA) transcript produced by a naturally-occurring organism. A recombinant nucleic acid may be produced using recombinant nucleic acid techniques. Recombinant nucleic acid techniques include techniques for constructing and manipulating the nucleotide sequences of nucleic acids, and include molecular cloning.

The term “transcriptional unit” refers to a DNA sequence that codes for a single RNA molecule (e.g. a mRNA molecule). A transcriptional unit comprises the sequence of nucleotides necessary for transcription, for example a transcriptional unit usually comprises a promoter, a polynucleotide sequence encoding a protein of interest, and a terminator sequence (such as a 3′ untranslated region also referred to as 3′-UTR).

The term “operably linked to” refers to the situation where nucleic acid encoding a recombinant polypeptide of interest, and regulatory nucleic acid sequence(s) (e.g. a polyadenylation signal, promoter, and/or enhancer) are covalently linked in such a way as to place the expression of the nucleic acid encoding a polypeptide of interest under the influence or control of the regulatory nucleic acid sequence(s) (thereby forming an transcriptional unit, or expression cassette). Thus, a (regulatory) sequence is operably linked to the selected nucleic acid sequence if the regulatory sequence is capable of effecting transcription of the nucleic acid sequence. The resulting transcript(s) may then be translated into the desired polypeptide(s) of interest.

The term “polyadenylation signal sequence” refers to a sequence that terminates transcription of a transcriptional unit and ensures that the nucleic acid sequence encoding a polypeptide is transcribed and translated properly. The polyadenylation signal is recognised by the RNA cleavage complex resulting in cleavage of the RNA and polyadenylation catalyzed by polyadenylate polymerase.

Examples of naturally-occurring eukaryotic polyadenylation signals include rabbit beta-globin poly(A) signal, a signal sequence that has been characterized in the literature as strong (Gil and Proudfoot, Cell 49: 399-406 (1987); Gil and Proudfoot, Nature 312: 473-474 (1984)). One of its key features is the structure of its downstream element, which contains both UG- and U-rich domains. Other polyadenylation signal sequences include synthetic polyA, HSV Thymidine kinase poly A, (see Cole, C. N. and T. P. Stacy, Mol. Cell. Biol. 5:2104-2113 (1985)); Human alpha globin poly A SV40 poly A (See Schek, N, Cooke, C, and J. C. Alwine, Mol. Cell Biol. 12:5386-5393 (1992)); human beta globin poly A (See Gil, A., and N. J. Proudfoot, Cell 49:399-406 (1987)); polyomavirus poly A (See Batt, D. B and G. G. Carmichael Mol. Cell. Biol. 15:4783-4790 (1995); Bovine growth hormone poly A, (Gimmi, E. R., Reff, M. E., and I. C. Deckman, Nucleic Acid Res. (1989)).

Additional polyadenylation sites can be identified or constructed using methods that are known in the art. A minimal polyadenylation site is composed of AAUAAA and a second recognition sequence, generally a G/U rich sequence, found about 30 nucleotides downstream. As used herein, the sequences are presented as DNA, rather than RNA, to facilitate preparation of suitable DNAs for incorporation into expression vectors. When presented as DNA, the polyadenylation site is composed of AATAAA, with, for example, a G/T rich region downstream. Both sequences must be present to form an efficient polyadenylation site. The purpose of these sites is to recruit specific RNA binding proteins to the RNA. The AAUAAA binds cleavage polyadenylation specificity factor (CPSF; Murthy K. G., and Manley J. L. (1995). Genes Dev 9:2672-2683), and second site, frequently a G/U sequence, binds to Cleavage stimulatory factor (CstF; Takagaki Y. and Manley J. L. (1997) Mol Cell Biol 17:3907-3914). CstF is composed of several proteins, but the protein responsible for RNA binding is CstF-64, a member of the ribonucleoprotein domain family of proteins (Takagaki et al. (1992) Proc Natl Acad Sci USA 89:1403-1407).

Without being bound to theory, it is appreciated that polyadenylation signal sequences belong to 3′ regulatory elements which are DNA sequences located in the 3′ untranslated region (UTR) of mRNA transcripts, downstream of the coding region. Other 3′ regulatory elements comprise AU-rich elements (AREs) and microRNA (miRNA) binding sites. 3′ regulatory elements are not translated into protein but play an important role in regulating gene expression, such as influencing the stability, localization, and translation of mRNA transcripts, and ultimately affect the expression (level) of protein-coding genes.

Provided herein are improved recombinant polyadenylation signal sequences that have several improved properties. The recombinant polyadenylation signal sequences of the present invention lead to improved expression (of a protein of interest which is encoded by a nucleotide sequence operably linked to the recombinant polyadenylation signal sequences). Additionally, the recombinant polyadenylation signal sequences of the present invention are shorter compared to effective polyadenylation signal sequences known in the art, for example compared to a polyadenylation signal sequence selected from the group consisting of the rabbit beta-globin poly(A) signal, HSV Thymidine kinase poly A, Human alpha globin poly A, SV40 poly A, human beta globin poly A, polyomavirus poly A, Bovine growth hormone poly A. In one embodiment, the recombinant polyadenylation signal sequence disclosed herein comprises less than 100, 99, 98, 97 or 96 nucleotides. In one embodiment, the recombinant polyadenylation signal sequence disclosed herein has a sequence length of less than 100, 99, 98, 97 or 96 nucleotides. In one embodiment, the recombinant polyadenylation signal sequence disclosed herein has a sequence length of between 25-100, between 30-100, between 35-100, between 40-100, between 50-100, between 55-100, between 60-100, between 65-100, between 70-100, between 75-100, between 80-100, between 85-100, between 90-100 nucleotides, or between 95-100 nucleotides. In one aspect, the recombinant polyadenylation signal sequence disclosed herein is recognized by a RNA polymerase whereupon the RNA polymerase releases the RNA molecule. In one aspect, the RNA polymerase is an eukaryotic RNA polymerase. In one aspect, a eukaryotic cell comprising a transcriptional unit comprising a nucleotide sequence encoding a polypeptide wherein the nucleotide sequence is operably linked to a recombinant polyadenylation signal sequence as herein disclosed is capable of expressing the polypeptide. In one aspect, the recombinant polyadenylation signal sequence leads to termination (of transcription) and polyadenylation of an mRNA transcript of the transcriptional unit in a eukaryotic cell. Hence, the recombinant polyadenylation signal sequence initiates termination of transcription of the transcriptional unit.

Without being bound to theory, it is advantageous in many applications of recombinant transcriptional units if regulatory elements are short. For example, viral vectors may be limited in size. Recombinant adeno-associated viruses (rAAV) are limited to a AAV transgene of below 5 kilobases and the transgene needs to include the coding sequences for the gene of interest along with promoter sequences, enhancers and polyadenylation signals. Short regulatory elements leave more space for coding sequences.

The term “expressing” means a process by which information from a nucleic acid is used in the synthesis of a functional polynucleotide enabling the production of a (gene) product, for example a protein of interest. Expression may include transcription, RNA splicing, translation and post-translational modifications. Regulation of expression gives control over the timing, location and amount of a given expression product (such as a protein of interest) present in a cell.

The term “termination” refers to the process by which RNA polymerase stops adding nucleotides to the growing RNA chain and releases the RNA molecule. “Polyadenylation” refers to the process by which a chain of adenine nucleotides, also referred to as a poly(A) tail, is added to the 3′ end of a newly synthesized RNA molecule. Polyadenylation is catalyzed by poly(A) polymerase and occurs after the RNA molecule has been cleaved at a specific site downstream of the coding region (polyadenylation signal). The term termination and polyadenylation refers to both processes in consecution to produce a mature mRNA transcript.

A “mRNA transcript”, also known a “messenger RNA” or “mRNA” refers to a type of RNA molecule that carries genetic information from the DNA in the nucleus of a cell to the ribosomes where it is used as a template to synthesize a protein. During the process of transcription, the DNA sequence of a protein-coding gene is used as a template to generate a complementary RNA molecule, which is processed and modified to form a mature mRNA transcript. The modifications to form a mature mRNA transcript include for example 5′ capping, splicing and polyadenylation.

In one embodiment, provided is a recombinant transcriptional unit comprising a nucleotide sequence encoding a polypeptide wherein the nucleotide sequence is operably linked to a recombinant polyadenylation signal sequence, wherein the recombinant polyadenylation signal sequence has a sequence length of less than 100 nucleotides. In one embodiment, a eukaryotic cell transformed with a recombinant nucleic acid comprising the recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2. In one embodiment, the nucleotide sequence of the reference nucleic acid is identical to the sequence of the recombinant nucleic acid not considering the recombinant polyadenylation signal sequence for sequence identity.

The term “expression level” refers to a quantitative determination of the level at which a particular open reading frame (such as included in a transcriptional unit) is expressed by a cell. The expression level can be determined for example by detecting the product of the open reading frame (such as a protein), by methods known in the art, such as for example Western blot analysis. However, it might be often easier to detect one of the precursors of the protein, such as mRNA and to infer gene-expression levels from these measurements. Levels of mRNA can be quantitatively measured by methods known in the art, such as for example northern blotting, RT-qPCR or a hybridization microarray. In one embodiment, the expression level is determined by RT-qPCR analysis. Another method to determine the expression level is the use of a reporter gene (also known as reporter) which is a gene that when operably liked to a regulatory sequence can be readily identified and measure for example by fluorescence or luminescence. Such reporter genes are well known in the art and also described herein.

For example in Example 2, a recombinant transcriptional unit is described including the luminescent protein NanoLuc luciferase. Expression levels of the luciferase can be determined by methods known in the art and as also shown in Example 1.3.

In one embodiment, the expression level effected by a recombinant transcriptional unit comprising a nucleotide sequence encoding a polypeptide wherein the nucleotide sequence is operably linked to a recombinant polyadenylation signal sequence is determined by:
(a) generating a reporter transcriptional unit by providing the nucleotide sequence of the recombinant transcriptional unit and replacing the nucleotide sequence encoding the polypeptide with the nucleotide sequence of SEQ ID NO:19 encoding for a luciferase,
(b) transfecting HEK293T cells with a reporter plasmid comprising the reporter transcriptional unit and culturing the HEK293T cell under conditions suitable for expression of the reporter transcriptional unit, and
(c) measuring the level of luciferase 24 h after transfection.

In one embodiment, the expression level is determined as described above for the recombinant nucleic acid to receive a first expression level, and then the expression level is determined as described above for the reference nucleic acid to receive a second expression level, followed by comparison of the first and second expression level to determine whether the first expression level is same or higher compared to the second expression level.

A “reference nucleic acid” as used herein refers to nucleic acid which is similar or identical to a recombinant nucleic acid of interest (such as a recombinant nucleic acid comprising a recombinant transcriptional unit of the present invention) apart from a sequence element of interest. The reference nucleic acid may be used to compare or benchmark functionality (e.g. effected expression level) of a recombinant nucleic acid of interest to a specific reference (e.g. to nucleic acid comprising a recombinant polyadenylation signal sequence consisting of the nucleotide sequence of SEQ ID NO:2). In some aspects, the nucleotide sequence of the reference nucleic acid is identical to the nucleotide sequence of the recombinant nucleic acid of interest apart from (not taking into account for sequence identity) a sequence element of interest such as for example a recombinant polyadenylation signal sequence of the invention. In some aspects, the nucleotide sequence of the recombinant polyadenylation signal sequence is not considered in the determination of the sequence identity. For example the nucleotide sequence of the recombinant polyadenylation signal sequence can be omitted (deleted) from the recombinant nucleic acid of interest and from the reference nucleic acid for the sequence comparison.

In one embodiment, a eukaryotic cell (individually) transformed with a recombinant nucleic acid comprising the recombinant transcriptional unit is capable of expressing the polypeptide at an expression level which is the same or higher compared to the expression level of the polypeptide in the (same type of) eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity. In one embodiment, the expression level is determined by (individually) integrating (each of) the recombinant polyadenylation signal sequence in a recombinant transcriptional unit comprising in 5′-3′ order a nucleotide sequence of SEQ ID NO:19 (encoding NanoLuc luciferase) and the recombinant polyadenylation signal sequence of interest, (individually) transfecting HEK293T cells with a reporter plasmid comprising the recombinant transcriptional unit, culturing the HEK293T cell under conditions suitable for expression of the recombinant transcriptional unit, and measuring the level of luciferase 24 h after transfection.

In one embodiment, the recombinant polyadenylation signal sequence comprises or consists of a nucleotide sequence having at least 90%, 91%, 92%, 93%, 94%; 95%, 96%, 97%, 98%, 99% or 100% sequence identity to a nucleotide sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

A particular aspect of the recombinant polyadenylation signal sequences according to the present invention is that they are functional in eukaryotic cells. For example, the recombinant polyadenylation signal sequences as herein provided are recognized by the RNA cleavage complex. In some aspects, the RNA cleavage complex is an eukaryotic RNA cleavage complex. In some embodiments, the recombinant polyadenylation signal sequence comprises TG- and T-rich domains. In some embodiments, the recombinant polyadenylation signal sequence comprises the nucleotide sequence AATAAA (SEQ ID NO:18). In some embodiments, the recombinant polyadenylation signal sequence comprises a G/T rich sequence about 30 nucleotides downstream of the nucleotide sequence AATAAA (SEQ ID NO:18). In some embodiments, the recombinant polyadenylation signal sequence when present in a RNA molecule is capable of binding to cleavage polyadenylation specificity factor (CPSF). In some embodiments, the recombinant polyadenylation signal sequence when present in a RNA molecule is capable of binding to cleavage stimulatory factor (CstF). In some embodiments, the at least one polyadenylation signal sequence leads to termination and polyadenylation of an mRNA transcript operably linked to the at least one polyadenylation signal sequence.

In some embodiments, provided is a recombinant transcriptional unit comprising a nucleotide sequence encoding a polypeptide wherein the nucleotide sequence is operably linked to a recombinant polyadenylation signal sequence, wherein the recombinant polyadenylation signal sequence comprises or consists of a nucleotide sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

In a preferred embodiment, the recombinant polyadenylation signal sequence comprises or consists of a nucleotide sequence selected from the group consisting of SEQ ID NO:6, SEQ ID NO:9, and SEQ ID NO:12.

In some embodiments, provided is recombinant nucleic acid comprising at least one polyadenylation signal sequence as provided herein. In case a plurality of a regulatory element (such as a polyadenylation signal sequence) is required in a recombinant nucleic acid (such as one or several plasmids), a common obstacle well known in the field is that identical sequences which come to close proximity can lead to recombination events. Hence, it is advantageous if such recombination events can be reduced or omitted. One aspect of the present invention are multiple new short recombinant polyadenylation signal sequences having (sharing) a low sequence homology (a low sequence identity) to one another. Furthermore, the recombinant polyadenylation signal sequences effect strong expression of a nucleotide sequence encoding a polypeptide of interest operably linked to the recombinant polyadenylation signal sequence.

In some embodiments, the recombinant nucleic acid comprises more than one recombinant polyadenylation signal sequence as provided herein. In some embodiments, the recombinant nucleic acid comprises two or more recombinant polyadenylation signal sequences as provided herein. In some embodiments, the recombinant nucleic acid comprises three recombinant polyadenylation signal sequences as provided herein. In a preferred embodiment, the recombinant nucleic acid comprises the polyadenylation signal sequences of SEQ ID NO:6, SEQ ID NO:9 and SEQ ID NO:12. In a particular such embodiment, the recombinant nucleic acid comprises three individual polyadenylation signal sequences, wherein the first polyadenylation signal sequence consists of the nucleotide sequence of SEQ ID NO:6, the second polyadenylation signal sequence consists of the nucleotide sequence of SEQ ID NO:9, and the third polyadenylation signal sequence consists of the nucleotide sequence of SEQ ID NO:12.

In one embodiment, provided is recombinant nucleic acid comprising:
(a) a first recombinant transcriptional unit comprising a first nucleotide sequence encoding a first polypeptide operably linked to a first recombinant polyadenylation signal sequence, and
(b) a second recombinant transcriptional unit comprising a second nucleotide sequence encoding a second polypeptide operably linked to a second recombinant polyadenylation signal sequence, wherein the first and second recombinant polyadenylation signal sequence have less than 70%, 65%, 60%, 55%, 50%, 45% or 40% sequence identity.

In one embodiment, the recombinant nucleic acid further comprises:
(c) a third recombinant transcriptional unit comprising a third nucleotide sequence encoding a third polypeptide operably linked to a third recombinant polyadenylation signal sequence, wherein the first and second recombinant polyadenylation signal sequence individually have less than 70%, 65%, 60%, 55%, 50%, 45% or 40% sequence identity with the third recombinant polyadenylation signal sequence.

In one embodiment, the recombinant nucleic acid further comprises:
(c) a third recombinant transcriptional unit comprising a third nucleotide sequence encoding a third polypeptide operably linked to a third recombinant polyadenylation signal sequence, wherein the first recombinant polyadenylation signal sequence has less than 70%, 65%, 60%, 55%, 50%, 45% or 40% sequence identity with the third recombinant polyadenylation signal sequence.

In one embodiment, the recombinant nucleic acid further comprises:
(c) a third recombinant transcriptional unit comprising a third nucleotide sequence encoding a third polypeptide operably linked to a third recombinant polyadenylation signal sequence, wherein the second recombinant polyadenylation signal sequence hase less than 70%, 65%, 60%, 55%, 50%, 45% or 40% sequence identity with the third polyadenylation signal sequence.

In some embodiments, the first, second, and where present third recombinant polyadenylation signal sequences have a sequence length of less than less than 100, 99, 98, 97 or 96 nucleotides. In some embodiments, the first, second, and where present third recombinant polyadenylation signal sequences have a sequence length of less than 100 nucleotides.

The term “percent (%) sequence identity” is defined as the percentage of nucleotides in a sequence of interest that are identical with the nucleotides in a candidate sequence, after aligning the sequences and introducing gaps, if necessary, to achieve the maximum percent sequence identity. Alignment can be achieved in various ways well known in the art; for instance, using publicly available software such as BLAST, BLAST-2, ALIGN, ALIGN-2 or Megalign (DNASTAR) software. Those skilled in the art can determine appropriate parameters for aligning sequences, including any algorithms needed to achieve maximal alignment over the full length of the sequences being compared.

In some aspects, percent (%) sequence identity of a first and second recombinant polyadenylation signal sequence is determined by:
(a) aligning the sequences of the first and second recombinant polyadenylation signal sequence using the Align-2 software and standard settings, and
(b) determining the percentage of nucleotides in the first recombinant polyadenylation signal sequence that are identical with the nucleotides in the second recombinant polyadenylation signal sequence to obtain the percent (%) sequence identity.

In some aspects, percent (%) sequence identity of a first and second recombinant polyadenylation signal sequence individually with a third recombinant polyadenylation signal sequence is determined by:
(a) aligning the sequences of the first and third recombinant polyadenylation signal sequence using the Align-2 software and standard settings,
(b) determining the percentage of nucleotides in the first recombinant polyadenylation signal sequence that are identical with the nucleotides in the third recombinant polyadenylation signal sequence to obtain the percent (%) sequence identity of the first and third recombinant polyadenylation signal sequences,
(c) aligning the sequences of the second and third recombinant polyadenylation signal sequence using the Align-2 software and standard settings,
(d) determining the percentage of nucleotides in the second recombinant polyadenylation signal sequence that are identical with the nucleotides in the third recombinant polyadenylation signal sequence to obtain the percent (%) sequence identity of the second and third recombinant polyadenylation signal sequences.

“Recombination events” between nucleic acid (e.g. plasmids) can occur through a variety of mechanisms, including homologous recombination and site-specific recombination. Such events can result in the transfer of genetic material from one plasmid to another or the integration of a plasmid into a chromosome. The resulting plasmids/chromosome may have different genetic content and may confer different function to the cell. In the context of the present invention, such recombination events are unwanted and the inventors sought to provide new and improved sequences to reduce or inhibit recombination events. Hence, in some aspects, recombination events between the nucleic acid comprising the first recombinant polyadenylation signal sequence and nucleic acid comprising the second recombinant polyadenylation signal sequence (and where present the third recombinant polyadenylation signal sequence) is reduced or prevented.

The present inventors have generated new and improved recombinant polyadenylation signal sequences which reduce/inhibit/prevent recombination events. The provided new and improved sequences are short and share a low degree of sequence identity. In some aspects, the first, second, and where present third polyadenylation signal sequence cannot engage in DNA strand exchange to form a recombination intermediate. “DNA strand exchange” is a critical step in the process of reciprocal recombination. Two DNA molecules break at corresponding positions and exchange segments of their strand with each other, before rejoining to form two new hybrid DNA molecules. DNA strand exchange involves the formation of a heteroduplex structure, in which the single-stranded ends of the broken polynucleotide molecules invade each other's double helix and form a region of base-pairing between the two molecules (Holliday junction). This “recombination intermediate” allows the DNA strands to cross over each other facilitating the exchange of DNA segments between the two molecules. Afterwards the Holliday junction can be resolved by cleavage of the strands, leading to the formation of hybrid DNA molecules.

The formation of a Holliday junction during homologous recombination requires a significant degree of sequence homology between the two DNA molecules involved in the exchange. Specifically, the homologous sequences must be long enough and have a high enough degree of similarity to form a stable heteroduplex DNA structure. Without being bound to theory, the minimum length and degree of homology required to form a Holliday junction can vary depending on the DNA molecules involved, as well as the specific enzymes and cofactors that are involved. In general, it is believed that a minimum of 100-200 base pairs of contiguous, homologous DNA sequence is required to form a stable Holliday junction.

In some aspects, recombination events between nucleic acid comprising the first polyadenylation signal sequence and nucleic acid comprising the second polyadenylation signal sequence are reduced or prevented. In some aspects, recombination events between nucleic acid comprising the first polyadenylation signal sequence and nucleic acid comprising the third polyadenylation signal sequence are reduced or prevented. In some embodiments, recombination events between nucleic acid comprising the second polyadenylation signal sequence and nucleic acid comprising the third polyadenylation signal sequence is reduced or prevented.

Recombination events can be detected with methods known in the art. For example, recombination events can be detected by Sanger sequencing of relevant PCR amplicons followed by alignment of the sequences (e.g. with CLUSTALW) and identification of recombination events (e.g. with Recombination Detection Program). In some aspects, recombination events are detected by Sanger sequencing of PCR amplicons of the nucleic acid comprising the recombinant polyadenylation signal sequences as herein provided followed by alignment of the PCR amplicons with CLUSTALW using standard settings and identification of recombination events with Recombination Detection Program 5 using standard settings. In preferred aspects, no recombination events are detected.

The recombinant polyadenylation signal sequences of the present invention are useful in different applications. Polyadenylation signal sequences are required for efficient protein expression. Hence, in some aspect, the recombinant transcriptional units according to the present invention are capable of driving expression of a nucleotide sequence encoding a polypeptide of interest. In some aspect, the nucleotide sequence encoding the polypeptide of interest is operably linked to the recombinant transcriptional unit. In some aspect, the nucleotide sequence encoding the polypeptide of interest is operably linked to the recombinant polyadenylation signal sequence as provided herein.

In some aspect, the first and second, and where present third, recombinant transcriptional unit is active in a eukaryotic cell. In some aspects, the first and second, and where present third, polypeptide is expressed by a eukaryotic cell. In some aspects, the eukaryotic cell is incubated under conditions suitable for expression of the first, second, and where present third, polypeptide. In some aspects, the eukaryotic cell is cultured under conditions suitable for expression of the first, second, and where present third, polypeptide. In some aspects, provided are methods for producing one (or several) polypeptides comprising the step of culturing a host cell comprising at least one recombinant transcriptional unit as herein described under conditions suitable for expression of the one (or several) polypeptides.

Protein expression can be measured by assays readily available in the art, such as described in the Examples provided herein below.

The terms plasmid, construct and vector are used throughout the specification. As used herein, the term “plasmid” refers to a circular, supercoiled DNA molecule into which various nucleic acid molecules coding for regulatory sequences, open reading frames, cloning sites, stop codons, spacer regions or other sequences selected for structural or functional regions are assembled and used as a vector to express genes in a vertebrate host. Further, as used herein, “plasmids” are capable of replicating in a bacterial strain. As used herein, the term “construct” refers to a particular vector or plasmid having a specified arrangement of genes and regulatory elements. A nucleic acid sequence can be “exogenous”, which means that it is foreign to the cell into which the vector is being introduced, “heterologous” which means that it is derived from a different genetic source or “homologous”, which means that the sequence is structurally related to a sequence in the cell but in a position within the host cell nucleic acid in which the sequence is ordinarily not found. Methods to construct a vector or modify a plasmid of the invention are well known in the art through standard recombinant techniques, which are described in for example in Sambrook et al. Molecular Cloning. A Laboratory Manual, Cold Spring Harbor Laboratory, New York, (1989) and Ausubel et a/., Current Protocols in Molecular Biology, Wiley Interscience Publishers, New York (1995) both incorporated herein by reference.

The term “vector” is used to refer to a carrier nucleic acid molecule into which a designated nucleic acid molecule encoding an antigen or antigens can be inserted for introduction into a cell where it can be expressed. Vectors include plasmids, cosmids, viruses (bacteriophage, animal viruses, and plant viruses). and artificial chromosomes (e.g., YACs). The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of expressed interfering RNA (eiRNA), short interfering RNA (siRNA), antisense molecules or ribozymes. Expression vectors can contain a variety of “control sequences”, which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described below.

It will be appreciated that to prevent recombination events within nucleic acid comprising multiple transcriptional units, apart from the recombinant polyadenylation signal sequences also further elements of the transcriptional units might form a recombination intermediate which could lead to a recombination event. Hence, it is preferred that the different recombinant transcriptional units do not comprise elements which share a high sequence homology. It will be further appreciated that, usually, the nucleotide sequences encoding the polypeptides of interest will not share a high sequence homology. However, in the situation that closely related genes encoding polypeptides of interest are included in different trancriptional units comprised in the nucleic acid as herein provided, it is preferred that the nucleotide sequences encoding the different polypeptides have less than 70%, 65%, 60%, 55%, 50%, 45% or 40% sequence identity. Further elements of the transcriptional units that could form a recombination intermediate are the promoters included in the transcriptional units. In some aspect, the nucleic acid comprises a first and a second promoter. In some aspect, the first and second promoter are not the same promoter. In some aspects, the nucleic acid further comprises a third promoter. In some aspects, the third promoter is not the same promoter as the first and/or second promoter.

In one embodiment, provided is recombinant nucleic acid as described herein before, wherein
(a) the first recombinant transcriptional unit further comprises a first promoter operably linked to the nucleotide sequence encoding a first polypeptide, and
(b) the second recombinant transcriptional unit further comprises a second promoter operably linked to the nucleotide sequence encoding a second polypeptide, wherein the first and second promoter have less than 70%, 65%, 60%, 55%, 50%, 45% or 40% sequence identity.

In one embodiment, the recombinant nucleic acid further comprises:
(c) where present the first recombinant transcriptional unit further comprises a first promoter operably linked to the nucleotide sequence encoding the first polypeptide,
wherein the first and second promoter have less than 80%, 79%, 78%, 77%, 76%, 75%, 74%, 73%, 72%, 71%,70%, 65%, or 60% sequence identity with the third promoter.

In one embodiment, provided is recombinant nucleic acid comprising:
(a) a first recombinant transcriptional unit comprising a first nucleotide sequence encoding a first polypeptide operably linked to a first recombinant polyadenylation signal sequence, wherein the first recombinant polyadenylation signal sequence comprises or consists of a nucleotide sequence having at least 90%, 91%, 92%, 93%, 94%; 95%, 96%, 97%, 98%, 99% or 100% sequence identity to the nucleotide sequences of SEQ ID NO:6, and
(b) a second recombinant transcriptional unit comprising a second nucleotide sequence encoding a second polypeptide operably linked to a second recombinant polyadenylation signal sequence, wherein the first recombinant polyadenylation signal sequence comprises or consists of a nucleotide sequence having at least 90%, 91%, 92%, 93%, 94%; 95%, 96%, 97%, 98%, 99% or 100% sequence identity to the nucleotide sequences of SEQ ID NO:9, optionally
wherein a eukaryotic cell transformed with a recombinant nucleic acid comprising the first recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity, and
wherein a eukaryotic cell transformed with a recombinant nucleic acid comprising the second recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity.

In one embodiment, provided is recombinant nucleic acid comprising:
(a) a first recombinant transcriptional unit comprising a first nucleotide sequence encoding a first polypeptide operably linked to a first recombinant polyadenylation signal sequence, wherein the first recombinant polyadenylation signal sequence comprises or consists of a nucleotide sequence having at least 90%, 91%, 92%, 93%, 94%; 95%, 96%, 97%, 98%, 99% or 100% sequence identity to the nucleotide sequences of SEQ ID NO:6, and
(b) a second recombinant transcriptional unit comprising a second nucleotide sequence encoding a second polypeptide operably linked to a second recombinant polyadenylation signal sequence, wherein the first recombinant polyadenylation signal sequence comprises or consists of a nucleotide sequence having at least 90%, 91%, 92%, 93%, 94%; 95%, 96%, 97%, 98%, 99% or 100% sequence identity to the nucleotide sequences of SEQ ID NO:12, optionally
wherein a eukaryotic cell transformed with a recombinant nucleic acid comprising the first recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity, and
wherein a eukaryotic cell transformed with a recombinant nucleic acid comprising the second recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity.

In one embodiment, provided is recombinant nucleic acid comprising:
(a) a first recombinant transcriptional unit comprising a first nucleotide sequence encoding a first polypeptide operably linked to a first recombinant polyadenylation signal sequence, wherein the first recombinant polyadenylation signal sequence comprises or consists of a nucleotide sequence having at least 90%, 91%, 92%, 93%, 94%; 95%, 96%, 97%, 98%, 99% or 100% sequence identity to the nucleotide sequences of SEQ ID NO:9, and
(b) a second recombinant transcriptional unit comprising a second nucleotide sequence encoding a second polypeptide operably linked to a second recombinant polyadenylation signal sequence, wherein the first recombinant polyadenylation signal sequence comprises or consists of a nucleotide sequence having at least 90%, 91%, 92%, 93%, 94%; 95%, 96%, 97%, 98%, 99% or 100% sequence identity to the nucleotide sequences of SEQ ID NO:12, optionally
wherein a eukaryotic cell transformed with a recombinant nucleic acid comprising the first recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity, and
wherein a eukaryotic cell transformed with a recombinant nucleic acid comprising the second recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity.

In one embodiment, provided is recombinant nucleic acid comprising:
(a) a first recombinant transcriptional unit comprising a first nucleotide sequence encoding a first polypeptide operably linked to a first recombinant polyadenylation signal sequence, wherein the first recombinant polyadenylation signal sequence comprises or consists of the nucleotide sequence of SEQ ID NO:6, and
(b) a second recombinant transcriptional unit comprising a second nucleotide sequence encoding a second polypeptide operably linked to a second recombinant polyadenylation signal sequence, wherein the first recombinant polyadenylation signal sequence comprises or consists of the nucleotide sequence having of SEQ ID NO:9.

In one embodiment, provided is recombinant nucleic acid comprising:
(a) a first recombinant transcriptional unit comprising a first nucleotide sequence encoding a first polypeptide operably linked to a first recombinant polyadenylation signal sequence, wherein the first recombinant polyadenylation signal sequence comprises or consists of the nucleotide sequence of SEQ ID NO:6, and
(b) a second recombinant transcriptional unit comprising a second nucleotide sequence encoding a second polypeptide operably linked to a second recombinant polyadenylation signal sequence, wherein the first recombinant polyadenylation signal sequence comprises or consists of the nucleotide sequence having of SEQ ID NO:12.

In one embodiment, provided is recombinant nucleic acid comprising:
(a) a first recombinant transcriptional unit comprising a first nucleotide sequence encoding a first polypeptide operably linked to a first recombinant polyadenylation signal sequence, wherein the first recombinant polyadenylation signal sequence comprises or consists of the nucleotide sequence of SEQ ID NO:9, and
(b) a second recombinant transcriptional unit comprising a second nucleotide sequence encoding a second polypeptide operably linked to a second recombinant polyadenylation signal sequence, wherein the first recombinant polyadenylation signal sequence comprises or consists of the nucleotide sequence having of SEQ ID NO:12.

In one embodiment, provided is recombinant nucleic acid comprising:
(a) a first recombinant transcriptional unit comprising a first nucleotide sequence encoding a first polypeptide operably linked to a first recombinant polyadenylation signal sequence, wherein the first recombinant polyadenylation signal sequence comprises or consists of a nucleotide sequence having at least 90%, 91%, 92%, 93%, 94%; 95%, 96%, 97%, 98%, 99% or 100% sequence identity to the nucleotide sequences of SEQ ID NO:6,
(b) a second recombinant transcriptional unit comprising a second nucleotide sequence encoding a second polypeptide operably linked to a second recombinant polyadenylation signal sequence, wherein the first recombinant polyadenylation signal sequence comprises or consists of a nucleotide sequence having at least 90%, 91%, 92%, 93%, 94%; 95%, 96%, 97%, 98%, 99% or 100% sequence identity to the nucleotide sequences of SEQ ID NO:9, and
(c) a third recombinant transcriptional unit comprising a third nucleotide sequence encoding a third polypeptide operably linked to a third recombinant polyadenylation signal sequence, wherein the third recombinant polyadenylation signal sequence comprises or consists of a nucleotide sequence having at least 90%, 91%, 92%, 93%, 94%; 95%, 96%, 97%, 98%, 99% or 100% sequence identity to the nucleotide sequences of SEQ ID NO:12, optionally
wherein a eukaryotic cell transformed with a recombinant nucleic acid comprising the first recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity,
wherein a eukaryotic cell transformed with a recombinant nucleic acid comprising the second recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity, and
wherein a eukaryotic cell transformed with a recombinant nucleic acid comprising the third recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity.

In one embodiment, provided is recombinant nucleic acid comprising:
(a) a first recombinant transcriptional unit comprising a first nucleotide sequence encoding a first polypeptide operably linked to a first recombinant polyadenylation signal sequence, wherein the first recombinant polyadenylation signal sequence comprises or consists of the nucleotide sequence of SEQ ID NO:6,
(b) a second recombinant transcriptional unit comprising a second nucleotide sequence encoding a second polypeptide operably linked to a second recombinant polyadenylation signal sequence, wherein the first recombinant polyadenylation signal sequence comprises or consists of the nucleotide sequence of SEQ ID NO:9, and
(c) a third recombinant transcriptional unit comprising a third nucleotide sequence encoding a third polypeptide operably linked to a third recombinant polyadenylation signal sequence, wherein the third recombinant polyadenylation signal sequence comprises or consists of the nucleotide sequence of SEQ ID NO:12

In some aspects, the first, second and where present third promoter is active in eukaryotic cells. In some aspects, the first, second and where present third promoter is capable of driving expression of the polypeptide(s) of interest in eukaryotic cells. Expression of the polypeptide of interest can be measured by assays readily available in the art, such as described in the Examples provided herein below. In some aspects, the first promoter drives expression of the first polypeptide. In some aspects, the second promoter drives expression of the second polypeptide. In some aspects, the third promoter drives expression of the third polypeptide. In some aspects, the first promoter is capable of driving expression of the first polypeptide and the second promoter is capable of driving expression of the second polypeptide, and where present the third promoter is capable of driving expression of the third polypeptide.

The term ,,promoter” denotes a polynucleotide sequence that controls transcription of a gene/structural gene or nucleic acid sequence to which it is operably linked. A promoter includes signals for RNA polymerase binding and transcription initiation. The used promoter will be functional in the cell in which expression of the selected structural gene is contemplated. A large number of promoters including constitutive, inducible and repressible promoters from a variety of different sources are well known in the art (and identified in databases such as GenBank) and are available as or within cloned polynucleotides (from, e.g., depositories such as ATCC as well as other commercial or individual sources).

Typically, a promoter is located in the 5′ non-coding or untranslated region of a gene, proximal to the transcriptional start site of the structural gene. Sequence elements within promoters that function in the initiation of transcription are often characterized by consensus nucleotide sequences. These elements include RNA polymerase binding sites, TATA sequences, CAAT sequences, differentiation-specific elements (DSEs), cyclic AMP response elements (CREs), serum response elements (SREs), glucocorticoid response elements (GREs), and binding sites for other transcription factors, such as CRE/ATF, AP2, SP1, cAMP response element binding protein (CREB) and octamer factors. If a promoter is an inducible promoter, then the rate of transcription increases in response to an inducing agent, such as a CMV promoter followed by two tet-operator site, the metallothionein and heat shock promoters. The rate of transcription is not regulated by an inducing agent if the promoter is a constitutively active promoter. Exemplary eukaryotic promoters that have been identified as strong promoters for expression are the SV40 early promoter, the adenovirus major late promoter, the mouse metallothionein-I promoter, the Rous sarcoma virus long terminal repeat, the Chinese hamster elongation factor 1 alpha (CHEF-1), human EF-1 alpha, ubiquitin, and human cytomegalovirus major-immediate-early promoter (hCMV MIE).

In some aspects, the first, second, and where present third promoter are individually selected from the group consisting of the SV40 early promoter, the adenovirus major late promoter, the mouse metallothionein-I promoter, the Rous sarcoma virus long terminal repeat, the Chinese hamster elongation factor 1 alpha (CHEF-1), human EF-1 alpha, ubiquitin, and human cytomegalovirus major-immediate-early promoter (hCMV MIE).

The nucleic acid according to the present invention may be comprised/contained in, a vector, or a plurality of vectors.

Accordingly, the present disclosure also provides a vector, or plurality of vectors, comprising the nucleic acid or plurality of nucleic acids according to the present invention. The vector may facilitate delivery of the nucleic acid(s) encoding one or several recombinant transcriptional units according to the present disclosure to a cell. The vector may be an expression vector comprising elements required for expressing a recombinant polypeptide according to the present disclosure. The vector may comprise elements facilitating integration of the nucleic acid(s) into the genomic DNA of the cell into which the vector is introduced.

Nucleic acids and vectors according to the present disclosure may be provided in purified or isolated form, i.e. from other nucleic acid, or naturally-occurring biological material.

A vector may be a vector for expression of the nucleic acid in the cell (i.e. an expression vector). Such vectors may include a promoter sequence operably linked to a nucleotide sequence encoding a recombinant polypeptide according to the present disclosure. A vector may also include a termination codon (i.e. 3′ in the nucleotide sequence of the vector to the nucleotide sequence encoding the recombinant polypeptide(s) and expression enhancers. Any suitable vectors, promoters, enhancers and termination codons known in the art may be used to express a peptide or polypeptide from a vector according to the present disclosure.

Vectors contemplated in connection with the present disclosure include DNA vectors, RNA vectors, plasmids (e.g. conjugative plasmids (e.g. F plasmids), non-conjugative plasmids, R plasmids, col plasmids, episomes), viral vectors (e.g. retroviral vectors, e.g. gammaretroviral vectors (e.g. murine Leukemia virus (MLV)-derived vectors, e.g. SFG vector), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 and Morgan and Boyerinas, Biomedicines (2016) 4:9, which are both hereby incorporated by reference in their entirety. In some embodiments, a vector according to the present disclosure is a lentiviral vector.

In some aspects, the vector may be a eukaryotic vector, i.e. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

In some aspects, the first, second, and/or where present third vector comprise a bacterial origin of replication. In some aspects, the first vector comprises a bacterial origin of replication. In some aspects, the second vector comprises a bacterial origin of replication. In some aspects, the third vector comprises a bacterial origin of replication. A bacterial origin of replication is required for replication of a vector, such as a plasmid, in bacteria. Bacterial origins of replication are know in the art. In some aspects, the bacterial original of replication is the pUC origin of replication.

In some aspects, the recombinant nucleic acid as herein provided comprises a first vector as described hereinabove comprising the first recombinant transcriptional unit as described herein above, and a second vector as described hereinabove comprising the second recombinant transcriptional unit as described herein above, and where a third recombinant transcriptional unit is present a third vector as described hereinabove comprising the third recombinant transcriptional unit as described hereinabove. The recombinant nucleic acid can be provided in one vial or several vials. For example, the first, second, and where present third vector might be provided together in one vial. Alternatively, the first, second, and where present third vector might be provided in separate vials. In some aspects, the first, second and where present third vector are provided in separate vials but and the vials together constitute the recombinant nucleic acid as herein provided. In some aspect, the first, second and where present third vector are provided in the same vial.

In some aspects, the recombinant nucleic acid comprises at least one selectable marker. In some aspects, the vector as described hereinabove comprises a selectable marker. The term ,,selectable marker” denotes a nucleic acid that allows cells carrying it to be specifically selected for or against, in the presence of a corresponding selection agent. Typically, a selectable marker will confer resistance to a drug or compensate for a metabolic or catabolic defect in the cell into which it is introduced. A selectable marker can be positive, negative, or bifunctional. A useful positive selectable marker is an antibiotic resistance gene allowing for the selection of cells transformed therewith in the presence of the corresponding selection agent, e.g. the antibiotic. A non-transformed cell is not capable to grow or survive under the selective conditions, i.e. in the presence of the selection agent. Negative selectable markers allow cells carrying the marker to be selectively eliminated. selectable markers used with eukaryotic cells include, e.g., the structural genes encoding aminoglycoside phosphotransferase (APH), such as e.g. the hygromycin (hyg), neomycin (neo), and G418 selectable markers, dihydrofolate reductase (DHFR), thymidine kinase (tk), glutamine synthetase (GS), asparagine synthetase, tryptophan synthetase (selection agent indole), histidinol dehydrogenase (selection agent histidinol D), and nucleic acids conferring resistance to puromycin, bleomycin, phleomycin, chloramphenicol, Zeocin, and mycophenolic acid.

In some aspects, the selectable marker is selected from the group consisting of the hygromycin selectable marker, the neomycin selectable marker, the G418 selectable markers, dihydrofolate reductase (DHFR), thymidine kinase, glutamine synthetase, asparagine synthetase, tryptophan synthetase, histidinol dehydrogenase, and nucleic acids conferring resistance to puromycin, bleomycin, phleomycin, chloramphenicol, Zeocin, and mycophenolic acid.

In some aspects, the recombinant nucleic acid comprises at least one bacterial origin of replication. In some aspects, the vector as described hereinabove comprises a bacterial origin of replication. In order for vectors/plasmids to replicate independently within a bacterial cell, they must possess a stretch of DNA that can act as an origin of replication. The origin of replication (also called the replication origin) is a particular sequence at which replication is initiated. Exemplary origin of replication can be derived from the pUC plasmid cloning vectors created by Joachim Messing and co-workers (Yanisch-Perron, C.; Vieira, J.; Messing, J. (1985). Gene. 33 (1): 103-119), In some aspects, the the first, second, and/or where present the third vector comprise a bacterial origin of replication, in particular the pUC19 origin of replication.

It will be appreciated that the nucleic acid of the present invention can used for recombinant production of proteins. As described herein before, use of the new recombinant polyadenylation signal sequences is advantageous in the context of recombinant polypeptide expression to mitigate the risk of recombination events between highly homologous or identical sequence stretches.

Accordingly, further provided is a cell (e.g. a host cell) comprising the recombinant nucleic acid according to the invention.

In some aspects, the cell is a host cell. The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

For recombinant production of a protein of interest, nucleic acids encoding a protein of interest are isolated and inserted into one or more vectors for further cloning and/or expression in a host cell. Such nucleic acids may be readily isolated and sequenced using conventional procedures or produced by recombinant methods or obtained by chemical synthesis.

Suitable host cells for cloning or expression of a protein of interest include prokaryotic or eukaryotic cells described herein. For example, (recombinant) polypeptides may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.). After expression, the protein of interest may be isolated from the bacterial cell paste in a soluble fraction and can be further purified.

In addition to prokaryotes, eukaryotic microbes such as filamentous fungi or yeast are suitable cloning or expression hosts for recombinant polypeptide-encoding vectors, including fungi and yeast strains whose glycosylation pathways have been “humanized”, resulting in the production of a polypeptide with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIESTM technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney (HEK) line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In some aspects, the (host) cell is an eukaryotic cell. In some aspects, the host cell is an eukaryotic host cell. In some aspects, the (host) cell is a mammalian (host) cell. In some aspects, the (host) cell is selected from the group consisting of CHO, BHK, HEK, and Sp2/0. In some aspect, the (host) cell is CHO K1.

Hence in one embodiment, provided is a method of producing a polypeptide comprising the steps of
(a) providing a host cell comprising the recombinant nucleic acid as described herein before,
(b) incubating the host cell under conditions suitable for expression of the polypeptide,
(c) recovering the polypeptide of interest from the cell culture.

In one embodiment, provided is a method of producing a polypeptide comprising the steps of
(a) providing a cell comprising the recombinant nucleic acid as described herein before comprising at least one polyadenylation signal sequence, wherein the at least one polyadenylation signal sequence is operably linked to a nucleotide sequence encoding the polypeptide,
(b) incubating the cell under conditions suitable for expression of the polypeptide,
(c) recovering the polypeptide of interest from the cell culture.

In one embodiment, provided is a method method of producing a polypeptide of interest, the method comprising the steps of
(a) providing a host cell comprising the recombinant nucleic acid as described herein before, wherein the polypeptide of interest is the first polypeptide, and wherein the second and where present third polypeptide are required for or improve the production of the polypeptide of interest,
(b) incubating the host cell under conditions suitable for expression of the first, second, and where present third polypeptide,
(c) recovering the polypeptide of interest from the cell culture, and optionally
(d) formulating the recovered polypeptide of interest for therapeutic use.

Further provided is the production of viral vectors using the recombinant polyadenylation signal sequences of the present invention. The production of viral vectors using multiple separate plasmids is a widely used and effective technique that allows for the generation of high-quality viral particles for research and clinical applications. However, since multiple plasmids are used, the potential for recombination events between highly homologous or identical sequence stretches on the different plasmids needs to be mitigated. The polyadenylation signal sequence according to the present invention are advantageous in this context. Furthermore, viral vector genomes are usually limited in size. Hence, it is advantageous to integrate short 5′ and 3′ regulatory sequences to maximize the sequence length available for the (therapeutic) transgene.

In some aspects, provided is a recombinant viral vector comprising a recombinant polyadenylation signal sequence as hereinbefore described.

In some aspects, provided is a recombinant viral vector comprising a capsid and a vector genome packages therein. In certain embodiments, viral vectors that may be used in the invention include, for example and without limitation, retroviral, adenoviral, helper-dependent adenoviral, hybrid adenoviral, herpes simplex virus, lentiviral, poxvirus, Epstein-Barr virus, vaccinia virus, and human cytomegalovirus vectors, including recombinant versions thereof. In a preferred embodiment, the recombinant viral vector comprises a lentiviral vector, an adenoviral vector or an adeno-associated (AAV) vector. In some aspect, the recombinant viral vector is a recombinant adeno-associated virus (rAAV) comprising an adeno-associated virus (AAV) capsid and a vector genome packaged therein.

I one embodiment, provided is a recombinant viral vector comprising a vector genome, wherein the vector genome comprises in 5′ to 3′ order:
(i) a 5′ ITR sequence,
(ii) a promoter sequence,
(iii) a sequence encoding a polypeptide,
(iv) a recombinant polyadenylation signal sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, and
(v) a 3′ ITR sequence.

In a preferred embodiment, the recombinant polyadenylation signal sequence is selected from the group consisting of SEQ ID NO:6, SEQ ID NO:9, and SEQ ID NO:12.

The term “recombinant”, as a modifier of a viral vector (such as a recombinant AAV (rAAV) vector), means that compositions have been manipulated (i.e., engineered) in a fashion that generally does not occur in nature. A particular example of a recombinant AAV vector would be where a nucleic acid that is not normally present in a wild-type AAV genome (heterologous polynucleotide) is inserted within a viral genome. An example of which would be where a nucleic acid (e.g., gene) encoding a therapeutic protein or polynucleotide sequence is cloned into a vector, with or without 5′, 3′ and/or intron regions that the gene is normally associated within the AAV genome. Although the term “recombinant” is not always used herein in reference to an AAV vector, recombinant forms are expressly included in spite of any such omission.

A “rAAV vector,” for example, is derived from a wild-type genome of AAV by using molecular methods to remove all or a part of a wild-type AAV genome, and replacing with a non-native (heterologous) nucleic acid, such as a nucleic acid encoding a therapeutic protein or polynucleotide sequence. Typically, for a rAAV vector one or both inverted terminal repeat (ITR) sequences of AAV genome are retained. A rAAV is distinguished from an AAV genome since all or a part of an AAV genome has been replaced with a non-native sequence with respect to the AAV genomic nucleic acid, such as with a heterologous nucleic acid encoding a therapeutic protein or polynucleotide sequence. Incorporation of a non-native (heterologous) sequence therefore defines an AAV as a “recombinant” AAV vector, which can be referred to as a “rAAV vector.”

In some aspects, a eukaryotic cell comprising the vector genome as hereinbefore described is capable of expressing the polypeptide. In some aspects, the recombinant polyadenylation signal sequence leads to termination (of transcription) and polyadenylation of an mRNA transcript of the transcriptional unit in a eukaryotic cell.

A recombinant AAV vector sequence can be packaged, referred to herein as a “particle” for subsequent infection (transduction) of a cell, ex vivo, in vitro or in vivo. Where a recombinant vector sequence is encapsidated or packaged into an AAV particle, the particle can also be referred to as a “rAAV,” “rAAV particle” and/or “rAAV virion”. Such rAAV, rAAV particles and rAAV virions include proteins that encapsidate or package a vector genome. Particular examples include in the case of AAV, capsid proteins.

A “vector genome”, which may be abbreviated as “vg”, refers to the portion of the recombinant plasmid sequence that is ultimately packaged or encapsidated to form a rAAV particle. In cases where recombinant plasmids are used to construct or manufacture recombinant AAV vectors, the AAV vector genome does not include the portion of the “plasmid” that does not correspond to the vector genome sequence of the recombinant plasmid. This non-vector genome portion of the recombinant plasmid is referred to as the “plasmid backbone”, which is important for cloning and amplification of the plasmid, a process that is needed for propagation and recombinant AAV vector production, but is not itself packaged or encapsidated into rAAV particles. Thus, a “vector genome” refers to the nucleic acid that is packaged or encapsidated by rAAV.

As used herein, the term “serotype” in reference to an AAV vector means a capsid that is serologically distinct from other AAV serotypes. Serologic distinctiveness is determined on the basis of lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes). An antibody to one AAV may cross-react with one or more other AAV serotypes due to homology of capsid protein sequence.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

rAAV viral vectors include any viral strain or serotype. For example and without limitation, a rAAV vector genome or particle (capsid, such as VP1, VP2 and/or VP3) can be based upon any AAV serotype, such as AAV-1, -2, -3, -4, -5, -6, -7, -8, -9, -10, -11, -12, -rh74, -rhlO, AAV3B or AAV-2i8, for example. Such vectors can be based on the same strain or serotype (or subgroup or variant), or be different from each other. For example and without limitation, a rAAV plasmid or vector genome or particle (capsid) based upon one serotype genome can be identical to one or more of the capsid proteins that package the vector. In addition, a rAAV plasmid or vector genome can be based upon an AAV serotype genome distinct from one or more of the capsid proteins that package the vector genome, in which case at least one of the three capsid proteins could be a different AAV serotype, e.g., AAV1, AAV2, AAV3, AAV4, AAV5, AAV6, AAV7, AAV8, AAV9, AAV10, AAV11, AAV12, -rh74, -rhlO, AAV3B, AAV-2i8, or variant thereof, for example. More specifically, a rAAV2 vector genome can comprise AAV2 ITRs but capsids from a different serotype, such as AAV1, AAV3, AAV4, AAV5, AAV6, AAV7, AAV8, AAV9, AAV10, AAV11, AAV12, -rh74, -rhlO, AAV3B, AAV-2i8, or variant thereof, for example. Accordingly, rAAV vectors include gene/protein sequences identical to gene/protein sequences characteristic for a particular serotype, as well as “mixed” serotypes, which also can be referred to as “pseudotypes.”

In certain embodiments, the rAAV plasmid or vector genome or particle is based upon reptile or invertebrate AAV variants, such as snake and lizard parvovirus (Penzes et al., 2015, J. Gen. Virol., 96:2769-2779) or insect and shrimp parvovirus (Roekring et al., 2002, Virus Res., 87:79-87).

In certain embodiments, the recombinant plasmid or vector genome or particle is based upon a bocavirus variant. Human bocavirus variants are described, for example, in Guido et al., 2016, World J. Gastroenterol., 22:8684-8697.

In one embodiment, the recombinant AAV (rAAV) vector comprises VP1, VP2, and/or VP3 capsid protein having 70% or more sequence identity to VP1, VP2 and/or VP3 capsid protein selected from the group consisting of AAV1, AAV2, AAV3, AAV4, AAV5, AAV6, AAV7, AAV8, AAV9, AAV 10, AAV11, AAV12, -rh74, -rhl0, AAV3B, AAV-2i8 VP1, VP2 and/or VP3 capsid protein. In one embodiment, the recombinant AAV (rAAV) vector comprises VP1, VP2, and/or VP3 capsid protein having 100% sequence identity to VP1, VP2 and/or VP3 capsid protein selected from the group consisting of AAV1, AAV2, AAV3, AAV4, AAV5, AAV6, AAV7, AAV8, AAV9, AAV 10, AAV11, AAV12, -rh74, -rhlO, AAV3B, AAV-2i8 VP1, VP2 and/or VP3 capsid protein. In certain embodiments, the AAV vector includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, AAV12, -rh74, -rhlO or AAV3B, ITR(s).

In certain embodiments, the recombinant AAV (rAAV) vectors include AAV1, AAV2, AAV3, AAV4,AAV5, AAV6, AAV7, AAV8, AAV9, AAV10, AAV11, AAV12, AAV3B, RhlO, Rh74 and AAV-2i8 variants (e.g., ITR and capsid variants, such as amino acid insertions, additions, substitutions and deletions) thereof, for example, as set forth in WO 2013/158879 (International Application PCT/US2013/037170), WO 2015/013313 (International Application PCT/US2014/047670) and US 2013/0059732 (U.S. application Ser. No. 13/594,773).

rAAV, such as AAV1, AAV2, AAV3, AAV4, AAV5, AAV6, AAV7, AAV8, AAV9, AAV 10, AAV11, AAV12, -rh74, -rh10, AAV3B, AAV-2i8 and variants, hybrids and chimeric sequences, can be constructed using recombinant techniques that are known to a skilled artisan, to include one or more heterologous polynucleotide sequences (transgenes) flanked with one or more functional AAV ITR sequences. Such AAV vectors typically retain at least one functional flanking ITR sequence(s), as necessary for the rescue, replication, and packaging of the recombinant vector into a rAAV vector particle. A rAAV vector genome would therefore include sequences required in cis for replication and packaging (e.g., functional ITR sequences).

In some aspects, the AAV capsid is selected from the group consisting AAV1, AAV2, AAV3, AAV4, AAV5, AAV6, AAV7, AAV8, AAV9, AAV 10, AAV11, AAV12, AAV-rh74, AAV-rh10, AAV3B, AAV-2i8 capsid or a variant capsid derived therefrom.

In some aspect, the recombinant adeno-associated virus (rAAV) comprises a vector genome comprising at least one promoter sequence. In some aspects, the promoter is selected from the group consisting of the SV40 early promoter, the adenovirus major late promoter, the mouse metallothionein-I promoter, the Rous sarcoma virus long terminal repeat, the Chinese hamster elongation factor 1 alpha (CHEF-1), human EF-1 alpha, ubiquitin, and human cytomegalovirus major-immediate-early promoter (hCMV MIE).

In further aspects, provided are methods of producing a recombinant adeno-associated virus (rAAV) vector.

In one embodiment, provided is a method of producing a recombinant adeno-associated virus (rAAV) vector, the method comprising the steps of
(a) providing a host cell comprising the recombinant nucleic acid as described herein before, wherein the first polynucleotide sequence encodes for a therapeutic payload, wherein the second nucleotide sequence encodes for viral vector rep and cap protein, wherein the third nucleotide sequence encodes for E4, E2a and VA protein,
(b) incubating the host cell under conditions suitable for production of the recombinant rAAV vector, and
(c) recovering the viral vector from the cell culture, and optionally
(d) formulating the recovered polypeptide of interest for therapeutic use.

Host cells in the context of producing a rAAV vector are used to replicate and package the viral genome genome into the AAV capsid. For example human embryonic kidney cells (HEK cells) that have been genetically engineered to produce the necessary proteins for AAV replication and capsid assembly are widely used to produce rAAV vectors. During the production of rAAV vectors, the host cells are usually transfected with multiple plasmids containing the AAV genome with the therapeutic gene, as well as the rep and cap gene required to replicate and package the viral genome into the capsid. The plasmids provide the necessary genetic material to produce the rAAV particles. The host cells replicate and package the AAV genome into the AAV particles which can then be harvested and purified, for example for use in gene therapy. Well-characterized host cells like HEK293 cells can help to ensure consistent and reliable production of rAAV particles. Other cells that can be used in the context of rAAV production are known in the art.

In some aspects, the host cell is an eukaryotic host cell. In some aspects, the host cell is a mammalian host cell. In some aspects, the host cell is selected from the group consisting of a CHO cell, a BHK cell, a HEK cell, and a Sp2/0 cell. In a preferred embodiment, the host cell is a HEK host cell, particularly a HEK293 host cell.

In some aspects, the polypeptides and rAAV vectors produced according to the present invention are further processed, such as for example formulated for therapeutic use. Hence, provided herein are also pharmaceutical compositions comprising a polypeptide produced according to the present invention or a rAAV vector produced according to the present invention. In one aspect, a pharmaceutical composition comprises any of the polypeptides or viral vectors provided herein and a pharmaceutically acceptable carrier. In another aspect, a pharmaceutical composition comprises any of the polypeptides or viral vectors provided herein and at least one additional therapeutic agent, e.g., as described below.

Pharmaceutical compositions (formulations) can be prepared by combining the polypeptide or viral vector with pharmaceutically acceptable carriers or excipients known to the skilled person. Exemplary pharmaceutical compositions as described herein are lyophilized, aqueous, frozen, etc.

Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

The pharmaceutical compositions to be used for in vivo administration are generally sterile. Sterility may be readily accomplished, e.g., by filtration through sterile filtration membranes.

Any of the polypeptides or viral vectors produced according to the present invention may be used in therapeutic methods.

In one aspect, an rAAV vector for use as a medicament is provided. In further aspects, a rAAV vector for use in treating a disease caused by the loss-of-function of a gene in a patient is provided. In certain aspects, a rAAV vector for use in a method of treatment is provided. In certain aspects, the invention provides a rAAV vector for use in a method of treating an individual having a loss-of-function genetic disease comprising administering to the individual an effective amount of the rAAV vector. A “loss-of-function genetic disease” refers to a type of genetic disorder in which a gene mutation or other genetic defect results in a reduced or absent production of a functional protein, leading to a disease phenotype. Therapies to treat such diseases are also referred to as gene replacement therapy in the field. Examples of such diseases include but are not limited to cystic fibrosis, sickle cell anemia, hemophilia and Tay-Sachs disease. In one such aspect, the method further comprises administering to the individual an effective amount of at least one additional therapeutic agent (e.g., one, two, three, four, five, or six additional therapeutic agents), e.g., as described below.

In a further aspect, the invention provides for the use of an rAAV vector in the manufacture or preparation of a medicament. In one aspect, the medicament is for treatment of a loss-of-function genetic disease. In a further aspect, the medicament is for use in a method of treating a loss-of-function genetic disease comprising administering to an individual having a loss-of-function genetic disease an effective amount of the medicament. In one such aspect, the method further comprises administering to the individual an effective amount of at least one additional therapeutic agent, e.g., as described below.

In a further aspect, the invention provides a method for treating a loss-of-function genetic disease. In one aspect, the method comprises administering to an individual having such loss-of-function genetic disease an effective amount of an rAAV vector. In one such aspect, the method further comprises administering to the individual an effective amount of at least one additional therapeutic agent, as described below.

An individual according to any of the above aspects is preferably a human.

In a further aspect, the invention provides pharmaceutical compositions comprising any of the rAAV vector provided herein, e.g., for use in any of the above therapeutic methods. In one aspect, a pharmaceutical composition comprises any of the rAAV vector provided herein and a pharmaceutically acceptable carrier. In another aspect, a pharmaceutical composition comprises any of the rAAV vector provided herein and at least one additional therapeutic agent, e.g., as described below.

rAAV vectors of the invention can be administered alone or used in a combination therapy. For instance, the combination therapy includes administering a rAAV vector of the invention and administering at least one additional therapeutic agent (e.g. one, two, three, four, five, or six additional therapeutic agents).

Such combination therapies noted above encompass combined administration (where two or more therapeutic agents are included in the same or separate pharmaceutical compositions), and separate administration, in which case, administration of the rAAV vector of the invention can occur prior to, simultaneously, and/or following, administration of the additional therapeutic agent or agents. In one aspect, administration of the rAAV vector and administration of an additional therapeutic agent occur within about one month, or within about one, two or three weeks, or within about one, two, three, four, five, or six days, of each other. In one aspect, the rAAV vector and additional therapeutic agent are administered to the patient on Day 1 of the treatment.

A rAAV vector produced according to the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

rAAV vectors produced according to the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The rAAV vector need not be, but is optionally formulated with one or more agents currently used to prevent or treat the disorder in question. The effective amount of such other agents depends on the amount of rAAV vector present in the pharmaceutical composition, the type of disorder or treatment, and other factors discussed above.

For the prevention or treatment of disease, the appropriate dosage of a rAAV vector produced according to the invention (when used alone or in combination with one or more other additional therapeutic agents) will depend on the type of disease to be treated, the type of rAAV vector, the severity and course of the disease, whether the rAAV vector is administered for preventive or therapeutic purposes, previous therapy, and the discretion of the attending physician. The rAAV vector is suitably administered to the patient at one time or over a series of treatments. The progress of this therapy is easily monitored by conventional techniques and assays.

In another aspect of the invention, an article of manufacture containing materials useful for the treatment, prevention and/or diagnosis of the diseases described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a rAAV vector produced according to the invention. The label or package insert indicates that the composition is used for treating the condition of choice. Moreover, the article of manufacture may comprise (a) a first container with a composition contained therein, wherein the composition comprises a rAAV vector produced according to the invention of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In the following statements, particular embodiments of the invention are described:

1. A recombinant transcriptional unit comprising a nucleotide sequence encoding a polypeptide wherein the nucleotide sequence is operably linked to a recombinant polyadenylation signal sequence, wherein the recombinant polyadenylation signal sequence has a sequence length of less than 100 nucleotides, and wherein a eukaryotic cell transformed with a recombinant nucleic acid comprising the recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity.

2. The recombinant transcriptional unit of embodiment 1, wherein the recombinant polyadenylation signal sequence comprises or consists of a nucleotide sequence having at least 90%, 91%, 92%, 93%, 94%; 95%, 96%, 97%, 98%, 99% or 100% sequence identity to a nucleotide sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

3. A recombinant transcriptional unit comprising a nucleotide sequence encoding a polypeptide wherein the nucleotide sequence is operably linked to a recombinant polyadenylation signal sequence, wherein the recombinant polyadenylation signal sequence comprises or consists of a nucleotide sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

4. The recombinant transcriptional unit of any one of embodiment 1-3, wherein the recombinant polyadenylation signal sequence comprises or consists of a nucleotide sequence selected from the group consisting of SEQ ID NO:6, SEQ ID NO:9, and SEQ ID NO:12.

5. Recombinant nucleic acid comprising:
(a) a first recombinant transcriptional unit comprising a first nucleotide sequence encoding a first polypeptide operably linked to a first recombinant polyadenylation signal sequence, and
(b) a second recombinant transcriptional unit comprising a second nucleotide sequence encoding a second polypeptide operably linked to a second recombinant polyadenylation signal sequence, wherein the first and second recombinant polyadenylation signal sequence have less than 70%, 65%, 60%, 55%, 50%, 45% or 40% sequence identity.

6. The recombinant nucleic acid of embodiment 5, further comprising:
(c) a third recombinant transcriptional unit comprising a third nucleotide sequence encoding a third polypeptide operably linked to a third recombinant polyadenylation signal sequence, wherein the first and second recombinant polyadenylation signal sequence individually have less than 70%, 65%, 60%, 55%, 50%, 45% or 40% sequence identity with the third recombinant polyadenylation signal sequence.

7. The recombinant nucleic acid of embodiment 5 or 6, wherein the first, second, and where present third recombinant polyadenylation signal sequence have a sequence length of less than 100 nucleotides.

8. The recombinant nucleic acid of embodiment 5-7, wherein the first, second, and where present third recombinant polyadenylation signal sequence cannot engage in DNA strand exchange to form a recombination intermediate.

9. The recombinant nucleic acid of any one of embodiments 5-8, wherein recombination events between the nucleic acid comprising the first recombinant polyadenylation signal sequence and nucleic acid comprising the second recombinant polyadenylation signal sequence are reduced or prevented.

10. The recombinant nucleic acid of any one of the embodiments 5-9, wherein recombination events between nucleic acid comprising the first recombinant polyadenylation signal sequence and nucleic acid comprising the third recombinant polyadenylation signal sequence are reduced or prevented, and/or wherein recombination events between nucleic acid comprising the second recombinant polyadenylation signal sequence and nucleic acid comprising the third recombinant polyadenylation signal sequence are reduced or prevented.

11. The recombinant nucleic acid of any one of embodiments 5-10, wherein the first, second, and where present third polypeptides are expressed in an eukaryotic cell.

12. The recombinant nucleic acid of any one of embodiments 5-11, wherein the first recombinant transcriptional unit is a recombinant transcriptional unit according to any one of embodiments 1-4, and wherein the second recombinant transcriptional unit is a recombinant transcriptional unit according to any one of embodiments 1-4, and wherein where present the third recombinant transcriptional unit is a recombinant transcriptional unit according to any one of embodiments 1-4.

13. The recombinant nucleic acid of any one of embodiments 5-12, wherein
(a) the first recombinant transcriptional unit further comprises a first promoter operably linked to the nucleotide sequence encoding the first polypeptide, and
(b) the second recombinant transcriptional unit further comprises a second promoter operably linked to the nucleotide sequence encoding the second polypeptide,
   wherein the first and second promoter have less than 70%, 65%, 60%, 55%, 50%, 45% or 40% sequence identity.

14. The recombinant nucleic acid of any one of embodiments 6-13, wherein:
(c) where present the first recombinant transcriptional unit further comprises a first promoter operably linked to the nucleotide sequence encoding the first polypeptide,
   wherein the first and second promoter have less than 80%, 79%, 78%, 77%, 76%, 75%, 74%, 73%, 72%, 71%,70%, 65%, or 60% sequence identity with the third promoter.

15. The recombinant nucleic acid of embodiment 13 or 14, wherein
(i) the first, second, and where present third promoters are active in eukaryotic cells,
(ii) the first promoter drives expression of the first polypeptide,
(iii) the second promoter drives expression of the second polypeptide,
(iv) the third promoter drives expression of the third polypeptide, and/or
(v) the thirst, second, and where present third promoter drive expression of the first, second, and where present third polypeptide, respectively.

16. The recombinant nucleic acid of any one of embodiments 13-15, wherein the first, second, and where present third promoter are individually selected from the group consisting of the hPGK1 promoter, the CMV promoter, and the hEF1α promoter.

17. The recombinant nucleic acid of any one of embodiments 5-16, wherein the recombinant nucleic acid comprises at least one vector.

18. The recombinant nucleic acid of any one of embodiments 5-17, wherein the recombinant nucleic acid comprises a first vector comprising the first recombinant transcriptional unit, and a second vector comprising the second recombinant transcriptional unit, and where a third recombinant transcriptional unit is present a third vector comprising the third recombinant transcriptional unit.

19. The recombinant nucleic acid of any one of embodiments 17 or 18, wherein the at least one vector comprises a selectable marker operably linked to the first, second, or where present third recombinant transcriptional unit, respectively.

20. The recombinant nucleic acid of embodiment 19, wherein the selectable marker is selected from the group consisting of the hygromycin selectable marker, the neomycin selectable marker, the G418 selectable markers, dihydrofolate reductase (DHFR), thymidine kinase, glutamine synthetase, asparagine synthetase, tryptophan synthetase, histidinol dehydrogenase, and nucleic acids conferring resistance to puromycin, bleomycin, phleomycin, chloramphenicol, Zeocin, and mycophenolic acid.

21. The recombinant nucleic acid of embodiment 17-20, wherein the first, second, and/or where present the third vector comprise a bacterial origin of replication, in particular the pUC19 origin of replication.

22. A host cell comprising the recombinant transcriptional unit of any one of embodiments 1-4 and/or the recombinant nucleic acid of any one of embodiments 5-21.

23. The host cell of embodiment 22, which is an eukaryotic host cell.

24. The host cell of embodiment 22 or 23, which is selected from the group consisting of CHO, BHK, HEK, and Sp2/0.

25. A recombinant viral vector comprising a vector genome, wherein the vector genome comprises in 5′ to 3′ order:
(i) a 5′ ITR sequence,
(ii) a promoter sequence,
(iii) a sequence encoding a polypeptide,
(iv) a recombinant polyadenylation signal sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, and
(v) a 3′ ITR sequence.

26. The recombinant viral vector of embodiment 25, wherein the recombinant polyadenylation signal sequence is selected from the group consisting of SEQ ID NO:6, SEQ ID NO:9, and SEQ ID NO:12.

27. The recombinant viral vector of embodiment 25 or 26, wherein the recombinant viral vector is selected from the group consisting of a retroviral vector, an adenoviral vector, a helper-dependent adenoviral vector, a hybrid adenoviral vector, a herpes simplex virus vector, a lentiviral vector, a poxvirus vector, an Epstein-Barr virus vector, a vaccinia virus vector, a human cytomegalovirus vectors, a lentiviral vector, an adenoviral vector or an adeno-associated virus (AAV) vector, or a recombinant variant derived therefrom.

28. The recombinant viral vector of any one of embodiments 25-27, wherein the recombinant viral vector is a recombinant adeno-associated virus (rAAV) vector.

29. The rAAV of embodiment 28, wherein the AAV capsid is selected from the group consisting AAV1, AAV2, AAV3, AAV4, AAV5, AAV6, AAV7, AAV8, AAV9, AAV 10, AAV11, AAV12, AAV-rh74, AAV-rh10, AAV3B, AAV-2i8 capsid or a variant capsid derived therefrom.

30. A method of producing a polypeptide of interest, the method comprising the steps of
(a) providing the host cell of any one of embodiments 22-24,
(b) incubating the host cell under conditions suitable for expression of the polypeptide,
(c) recovering the polypeptide of interest from the cell culture.

31. A method of producing a polypeptide of interest, the method comprising the steps of
(a) providing a host cell comprising the recombinant nucleic acid of any one of embodiments 4-21, wherein the polypeptide of interest is the first polypeptide, and wherein the second and where present third polypeptide are required for or improve the production of the polypeptide of interest,
(b) incubating the host cell under conditions suitable for expression of the first, second, and where present third polypeptide,
(c) recovering the polypeptide of interest from the cell culture, and optionally
(d) formulating the recovered polypeptide of interest for therapeutic use.

32. A method of producing a recombinant adeno-associated virus (rAAV) vector, the method comprising the steps of
(a) providing a host cell comprising the recombinant nucleic acid of any one of embodiments 4-21, wherein the first polynucleotide sequence encodes for a therapeutic payload, wherein the second nucleotide sequence encodes for viral vector rep and cap protein, wherein the third nucleotide sequence encodes for E4, E2a and VA protein,
(b) incubating the host cell under conditions suitable for production of the recombinant rAAV vector, and
(c) recovering the viral vector from the cell culture, and optionally
(d) formulating the recovered polypeptide of interest for therapeutic use.

33. The method of any one of embodiments 30-32, wherein the host cell is selected from the group consisting of a CHO cell, a BHK cell, a HEK cell, and a Sp2/0 cell.

34. The method of embodiment 32 or 33, wherein the rAAV vector is selected from the group consisting of AAV1, AAV2, AAV3, AAV4, AAV5, AAV6, AAV7, AAV8, AAV9, AAV 10, AAV11, AAV12, AAV-rh74, AAV-rh10, AAV3B, AAV-2i8 vector, or a vector variant derived therefrom.

35. Use of a recombinant transcriptional unit for recombinant production of a polypeptide of interest, wherein the recombinant transcriptional unit is as defined in any one of embodiments 1-4.

35. Use of a recombinant nucleic acid for recombinant production of a polypeptide of interest, wherein the recombinant nucleic acid is as defined in any one of embodiments 5-21.

36. The invention as hereinbefore described with reference to the Examples and Figures included herein.

### Exemplary Sequences

| | | SEQ ID |
|---|---|---|
| Construct name | Sequence | NO |
| polyA-1.1 (69 | TGAGCATCTGACTTCTGGCTAATAAAATATCTTTATTTAGC | 1 |
| nt) | ATACATCTGTGTGTTGGTTTTTTGTGTG | |
| polyA-1.2 (95 | TATTGACTGCTCTGAGAAAGTTGATTTGAGCATCTGACTTC | 2 |
| nt) | TGGCTAATAAAATATCTTTATTTAGCATACATCTGTGTGTT | |
| | GGTTTTTTGTGTG | |
| polyA-1.3 (95 | TATTGACTGCTCTGAGAAAGTTGATTTGAGCATCTGACTTC | 3 |
| nt) | TGGCTAATAAAATATCTTTATTTAGTCAACATCTGTGTGTT | |
| | GGTTTTTTGTGTG | |
| polyA-2.1 (69 | TGAACATCTGATGGTCTTTGAATAAAGTCTGAGTGAGTGGC | 4 |
| nt) | ATACATCTGTGTGTTGGTTTTTTGTGTG | |
| polyA-2.2 (95 | ACAGACAGTATTGCTTACGAGTTGATTGAACATCTGATGGT | 5 |
| nt) | CTTTGAATAAAGTCTGAGTGAGTGGCATACATCTGTGTGTT | |
| | GGTTTTTTGTGTG | |
| polyA-2.3 (95 | ACAGACAGTATTGCTTACGAGTTGATTGAACATCTGATGGT | 6 |
| nt) | CTTTGAATAAAGTCTGAGTGAGTGGTCAACATCTGTGTGTT | |
| | GGTTTTTTGTGTG | |
| polyA-3.1 (69 | TGAGCATCTCCTTTAATCATAATAAAATCTGTGATTTCTAG | 7 |
| nt) | CAACATCTGTGTGTTGGTTTTTTGTGTG | |
| polyA-3.2 (95 | ATCCAGTCGTGTAGTTCTTATTACCTTGAGCATCTCCTTTAA | 8 |
| nt) | TCATAATAAAATCTGTGATTTCTAGCAACATCTGTGTGTTG | |
| | GTTTTTTGTGTG | |
| polyA-3.3 (95 | ATCCAGTCGTGTAGTTCTTATTACCTTGAGCATCTCCTTTAA | 9 |
| nt) | TCATAATAAAATCTGTGATTTCTACAGACATCTGTGTGTTG | |
| | GTTTTTTGTGTG | |
| polyA-4.1 (69 | TGAACATCTGATAGTAAATTAATAAAAACTGTGTGAAGTG | 10 |
| nt) | CATACATCTGTGTGTTGGTTTTTTGTGTG | |
| polyA-4.2 (95 | ATCACGGCACTACACTCGTTGCTTTATGAACATCTGATAGT | 11 |
| nt) | AAATTAATAAAAACTGTGTGAAGTGCATACATCTGTGTGTT | |
| | GGTTTTTTGTGTG | |
| polyA-4.3 (95 | ATCACGGCACTACACTCGTTGCTTTATGAACATCTGATAGT | 12 |
| nt) | AAATTAATAAAAACTGTGTGAAGTGTCAACATCTGTGTGTT | |
| | GGTTTTTTGTGTG | |
| Levitt et al | AATAAAATATCTTTATTTTCATTACATCTGTGTGTTGGTTTT | 13 |
| (34 nt) | TTGTGTG | |
| 2xsnRP1 (49 nt) | AAATAAAATACGAAATGAAATAAAATACGAAATG | 14 |
| SV40 (122 nt) | TACTTGTTTATTGCAGCTTATAATGGTTACAAATAAAGCAA | 15 |
| | TAGCATCACAAATTTCACAAATAAAGCATTTTTTTCACTGC | |
| | ATTCTAGTTGTGGTTTGTCCAAACTCATCAATGTATCTTA | |
| BGH (208 nt) | CTGTGCCTTCTAGTTGCCAGCCATCTGTTGTTTGCCCCTCCC | 16 |
| | CCGTGCCTTCCTTGACCCTGGAAGGTGCCACTCCCACTGTC | |
| | CTTTCCTAATAAAATGAGGAAATTGCATCGCATTGTCTGAG | |
| | TAGGTGTCATTCTATTCTGGGGGGTGGGGTGGGGCAGGAC | |
| | AGCAAGGGGGAGGATTGGGAAGAGAATAGCAGGCATGCT | |
| | GGGGA | |
| hGH (477 nt) | GGGTGGCATCCCTGTGACCCCTCCCCAGTGCCTCTCCTGGC | 17 |
| | CCTGGAAGTTGCCACTCCAGTGCCCACCAGCCTTGTCCTAA | |
| | TAAAATTAAGTTGCATCATTTTGTCTGACTAGGTGTCCTTCT | |
| | ATAATATTATGGGGTGGAGGGGGGTGGTATGGAGCAAGGG | |
| | GCAAGTTGGGAAGACAACCTGTAGGGCCTGCGGGGTCTAT | |
| | TGGGAACCAAGCTGGAGTGCAGTGGCACAATCTTGGCTCA | |
| | CTGCAATCTCCGCCTCCTGGGTTCAAGCGATTCTCCTGCCT | |
| | CAGCCTCCCGAGTTGTTGGGATTCCAGGCATGCATGACCAG | |
| | GCTCAGCTAATTTTTGTTTTTTTGGTAGAGACGGGGTTTCAC | |
| | CATATTGGCCAGGCTGGTCTCCAACTCCTAATCTCAGGTGA | |
| | TCTACCCACCTTGGCCTCCCAAATTGCTGGGATTACAGGCG | |
| | TGAACCACTGCTCCCTTCCCTGTCCTT | |
| polyA signal | AATAAA | 18 |
| Luciferase | ATGGTCTTCACACTCGAAGATTTCGTTGGGGACTGGCGACA | 19 |
| sequence | GACAGCCGGCTACAACCTGGACCAAGTCCTTGAACAGGGA | |
| | GGTGTGTCCAGTTTGTTTCAGAATCTCGGGGTGTCCGTAAC | |
| | TCCGATCCAAAGGATTGTCCTGAGCGGTGAAAATGGGCTG | |
| | AAGATCGACATCCATGTCATCATCCCGTATGAAGGTCTGAG | |
| | CGGCGACCAAATGGGCCAGATCGAAAAAATTTTTAAGGTG | |
| | GTGTACCCTGTGGATGATCATCACTTTAAGGTGATCCTGCA | |
| | CTATGGCACACTGGTAATCGACGGGGTTACGCCGAACATG | |
| | ATCGACTATTTCGGACGGCCGTATGAAGGCATCGCCGTGTT | |
| | CGACGGCAAAAAGATCACTGTAACAGGGACCCTGTGGAAC | |
| | GGCAACAAAATTATCGACGAGCGCCTGATCAACCCCGACG | |
| | GCTCCCTGCTGTTCCGAGTAACCATCAACGGAGTGACCGGC | |
| | TGGCGGCTGTGCGAACGCATTCTGGCG | |

The present disclosure includes the combination of the aspects and preferred features described except where such a combination is clearly impermissible or expressly avoided.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in the specification and the appended claims, the singular forms “a”, “an”, and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about”, it will be understood that the particular value forms another embodiment.

Where a nucleic acid sequence is disclosed herein, the reverse complement thereof is also expressly contemplated.

Methods described herein may preferably be performed in vitro. The term “in vitro” is intended to encompass procedures performed with cells in culture whereas the term “in vivo” is intended to encompass procedures with/on intact multi-cellular organisms.

### EXAMPLES

The following are examples of methods and compositions of the invention. It is understood that various other embodiments may be practiced, given the general description provided above.

### Example 1

### Materials and Methods

### 1.1 Gene Synthesis

Desired gene segments and plasmids where required were synthesized by GenScript Biotech (Rijswijk, Netherlands).

### 1.2 Cell Culture and Transfection of Human Embryonic Kidney Cells (HEK293T)

HEK293T cells were cultured in DMEM (high glucose, GlutaMAX, pyruvate; Gibco, Cat. no. 31966) supplemented with 10% (v/v) fetal bovine serum (Gibco, Cat. no. A5209402) and 50 U/mL Penicillin-Streptomycin (Gibco, Cat. no. 15070063) and were routinely passaged using 0.25% Trypsin-EDTA (Gibco, Cat. no. 25200).

For transient transfection of HEK293T cells, 2500 cells in 20 μL per well were seeded into 384-well plates 1 day prior to transfection. Each well was then transiently transfected with a 5 μL transfection mixture consisting of 25 ng plasmid DNA that had been complexed with 0.05 μL Lipofectamine 2000 (Invitorgen. Cat. no. 11668019) for 20 min at room temperature in Opti-MEM reduced serum media (Gibco, Cat. no. 31985). All reporter plasmid used to assess the polyadenylation signals was transfected in equimolar amounts and the total amount of transfected plasmids per experimental condition was normalized to 25 ng using a mock plasmid devoid of active transcription and open reading frames.

### 1.3 Quantification of NanoLuc Luciferase (Nluc) Production

Total Nluc production was quantified using the Nano-Glo Luciferase Assay System (Promega, Cat. no. N1110) by adding 25 μL 2× Luciferase Assay Solution (1 Vol Nano-Glo Luciferase Assay Substrate mixed with 50 Vol Assay Buffer) to each well in a 384-well plate. Assay plates were incubated in the dark for 10 min at room temperature before quantifying Luminescence using a PHERAstar FSX (BMG Labtech) plate reader.

### Example 2

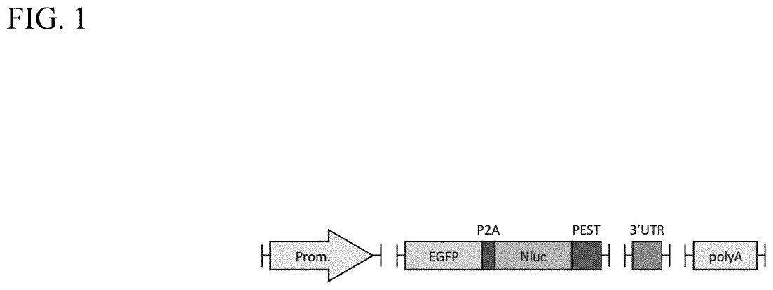

DNA sequences encoding a constitutive promoter (Prom.), enhanced green fluorescent protein (EGFP), P2A self-cleaving peptide sequence, NanoLuc luciferase (Nluc), PEST protein degradation signal, and a 3′ untranslated region (3′UTR), were used to assemble a transient transfection reporter plasmid for assessing the ability of polyadenylation (polyA) signal to support high expression levels through efficient transcriptional termination and polyadenylation (FIG. 1).

The standard BGH polyadenylation signal sequence and two copies of the short sNRP-1 polyadenylation signal sequence (2× sNRP-1; McFarland et al. (2006)) were inserted in the reporter plasmid directly downstream of the 3′UTR, respectively (FIG. 2A). To assess the recombinant polyadenylation signal sequence's relative transcriptional termination efficiency and ability to support high protein expression levels, HEK293T cells were transiently transfected with the corresponding reporter plasmid for 24 h and then assayed for total Nluc expression levels. FIG. 2B shows the relative Nluc expression levels from the two reporter plasmids, with the results normalized to the mean luminescence value of the cell transfected with the BGH encoding reporter plasmid. Notably, the short 2× sNRP-1 polyA encoding construct express <25% compared to the BGH encoding construct, highlighting how short polyadenylation signal sequence known in the art are less reliable for supporting high expression levels of a gene of interest.

### Example 3

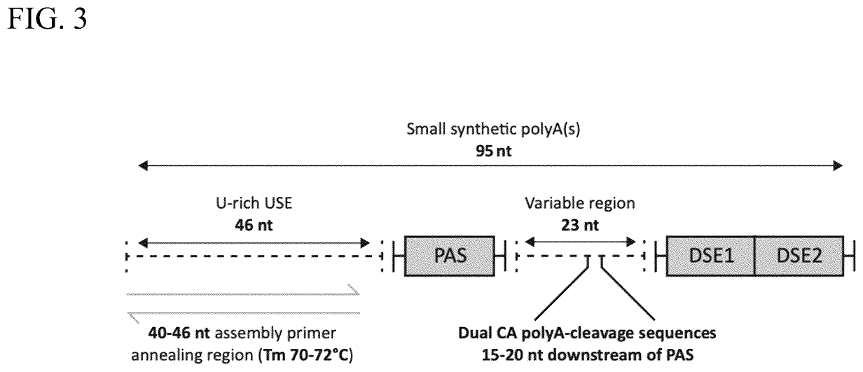

To establish a set of short recombinant polyadenylation signal sequences that can support high expression levels while having a high sequence heterogeneity to enable the use in multi-gene expression vectors without the risk of recombination, a 95 nucleotide (nt) recombinant polyadenylation signal sequence design was created consisting of the core elements of a polyadenylation signal sequence derived from a synthetic rabbit beta-globulin polyadenylation signal sequence defined by Levitt et al. (Levitt et al. (1989)), including polyadenylation signal (PAS), and two GU/U-rich downstream sequence element (DSE) regions. In addition, two cytosine-adenine (CA) mRNA cleavage sites were introduced 15-20 nt downstream of the PAS and a 26 nt U-rich upstream sequence element (USE) region was introduced (FIG. 3).

Based on the above-outlined polyA design, an initial set of four recombinant polyadenylation signal sequences were designed (polyA-1.1, -2.1, -3.1, and -4.1). Each of the four recombinant polyadenylation signal sequences were subsequently rationally modified by extending the USE region to 46 nt and designed to contain a unique primer annealing site with a Tm of 70-72° C. compatible with Gibson Assembly. Moreover, small nt modifications were introduced, focusing on the USE or the variable region between the PSA and DSE regions to increase heterogeneity between the polyA(s) or decrease strong secondary RNA structures within the polyA sequence. 12 recombinant polyadenylation signal sequences were selected and introduced into the above-described reporter plasmids (FIG. 4A), and transiently tested in HEK293T. FIG. 4B shows the relative Nluc expression levels from the reporter plasmids encoding the 12 recombinant polyadenylation signal sequences 24 h after transfection, respectively, with the results normalized to the mean luminescence values of all transfection conditions. From the tested set of recombinant polyadenylation signal sequences, three (polyA-2.3, -3.3, and -4.3) were selected for further characterization due to their ability to sustain higher relative expression levels and possessing <50% identical sequence compared to each other.

### Example 4

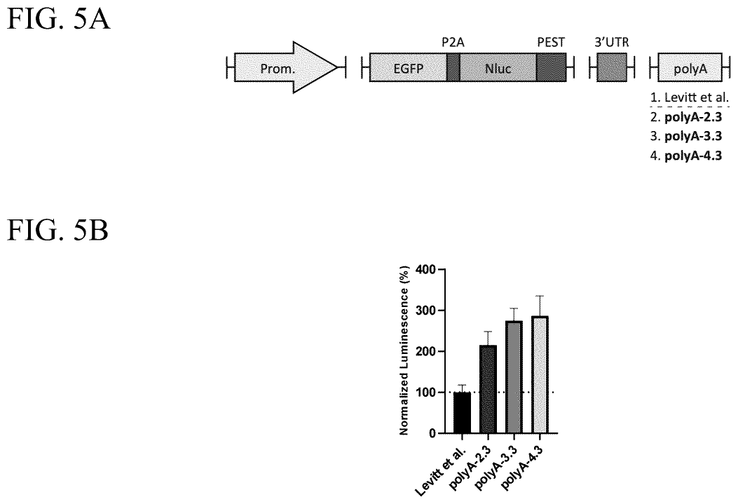

To benchmark the selected three recombinant polyadenylation signal sequences, the rabbit beta-globulin polyadenylation signal sequence, as defined by Levitt et al. (Levitt et al. (1989)), was introduced into the reporter plasmid (FIG. 5A) and transiently tested in HEK293T. FIG. 5B shows the relative Nluc expression levels from the Levitt et al. polyA and the three synthetic polyA encoding plasmids 24 h after transfection, respectively, with the results normalized to the mean luminescence value of the cell transfected with the Levitt et al. polyA encoding reporter plasmid. Notably, all three synthetic polyA encoding construct express greater than 2 times higher Nluc expression levels than the Levitt et al. rabbit beta-globulin polyadenylation signal sequence.

### Example 5

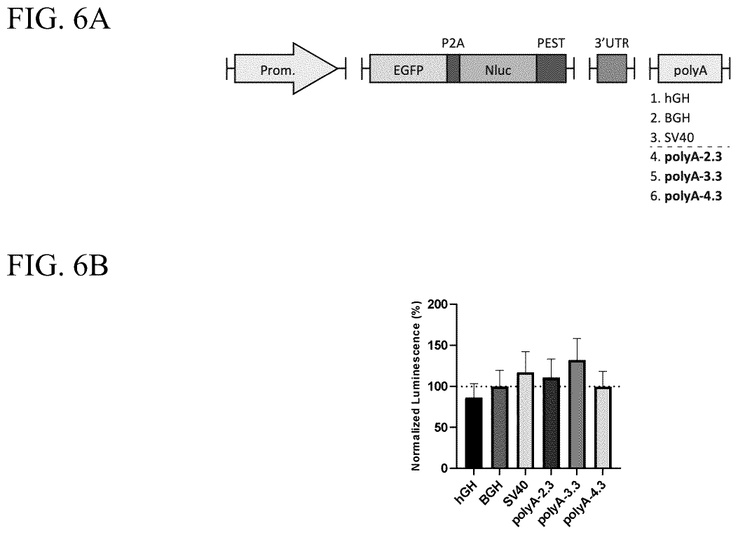

To benchmark the selected three recombinant polyadenylation signal sequences against known larger polyadenylation signal sequences, hGH polyA and SV40 polyA were introduced into the reporter construct and tested with the BGH-containing reporter plasmid, as mentioned above, against the recombinant polyadenylation signal sequence containing plasmids (FIG. 6A). To assess the recombinant polyadenylation signal sequence's relative transcriptional termination efficiency and ability to support high protein expression levels, HEK293T cells were transiently transfected with the corresponding reporter plasmid for 24 h and then assayed for total Nluc expression levels. FIG. 6B shows the relative Nluc expression levels from the reporter plasmids. The results are normalized to the mean luminescence value of the cell transfected with the BGH encoding reporter plasmid. Notably, the recombinant polyadenylation signal sequences support comparable or higher expression levels as the known larger polyadenylation signal sequences.

### Example 6

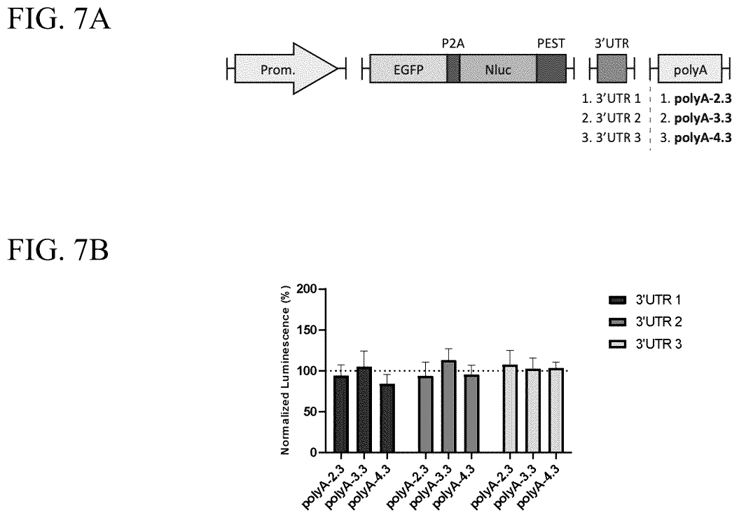

To assess how the recombinant polyadenylation signal sequences are affected by the upstream 3′UTR sequence composition, three different de novo designed 3′UTR sequences that share <50% pairwise sequence identity and <30% identical sequences were introduced into recombinant polyadenylation signal sequence encoding reporter plasmids (FIG. 7A). All constructs were transiently tested in HEK293T, as shown in FIG. 7B, depicting the relative Nluc expression levels from the reporter plasmids 24 h after transfection, with the results normalized to the mean luminescence values of all transfection conditions. Notably, the recombinant polyadenylation signal sequences demonstrate a minimal influence from the three different upstream 3′UTR sequences.

### Example 7

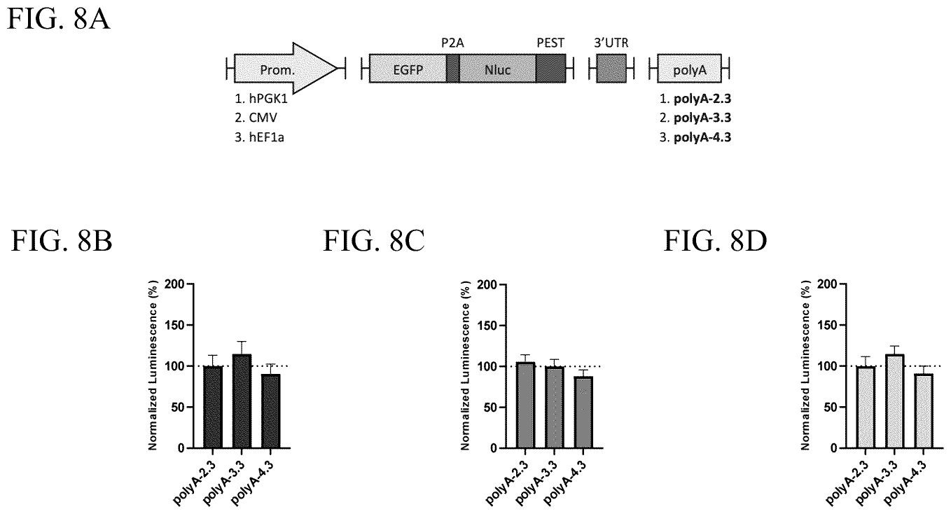

To assess how the recombinant polyadenylation signal sequences are affected by the strength of the promoter used and consequently the resulting expression levels, three constitutive promoters of different strengths, including hPGK1, CMV, and hEF1α, were introduced into the respective recombinant polyadenylation signal sequence encoding reporter plasmids (FIG. 8A). All constructs were transiently tested in HEK293T, and the relative Nluc expression levels from the reporter plasmids were quantified 24 h after transfection. FIG. 8B (hPGK1), FIG. 8C (CMV), and FIG. 8D (hEF1α), respectively, show relative Nluc expression levels from the different reporter plasmids, with the results normalized to the mean luminescence values of all transfection conditions in the corresponding graph. Notably, the recombinant polyadenylation signal sequences support robust expression at different levels in combination with different constitutive promoters.

1. A recombinant transcriptional unit comprising a nucleotide sequence encoding a polypeptide wherein the nucleotide sequence is operably linked to a recombinant polyadenylation signal sequence, wherein the recombinant polyadenylation signal sequence has a sequence length of less than 100 nucleotides, and wherein a eukaryotic cell transformed with a recombinant nucleic acid comprising the recombinant transcriptional unit is capable of expressing the polypeptide at an expression level which is the same or higher compared to the expression level of the polypeptide in the eukaryotic cell transformed with a reference nucleic acid comprising a recombinant polyadenylation signal sequence consisting of SEQ ID NO:2, wherein the nucleotide sequence of the reference nucleic acid is identical to the sequence of the recombinant nucleic acid not considering the recombinant polyadenylation signal sequence for sequence identity.

2. The recombinant transcriptional unit of claim 1, wherein the recombinant polyadenylation signal sequence comprises or consists of a nucleotide sequence having at least 90%, 91%, 92%, 93%, 94%; 95%, 96%, 97%, 98%, 99% or 100% sequence identity to a nucleotide sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

3. A recombinant transcriptional unit comprising a nucleotide sequence encoding a polypeptide wherein the nucleotide sequence is operably linked to a recombinant polyadenylation signal sequence, wherein the recombinant polyadenylation signal sequence comprises or consists of a nucleotide sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

4. The recombinant transcriptional unit of any one of claim 1-3, wherein the recombinant polyadenylation signal sequence comprises or consists of a nucleotide sequence selected from the group consisting of SEQ ID NO:6, SEQ ID NO:9, and SEQ ID NO:12.

5. Recombinant nucleic acid comprising:
(a) a first recombinant transcriptional unit comprising a first nucleotide sequence encoding a first polypeptide operably linked to a first recombinant polyadenylation signal sequence, and
(b) a second recombinant transcriptional unit comprising a second nucleotide sequence encoding a second polypeptide operably linked to a second recombinant polyadenylation signal sequence, wherein the first and second recombinant polyadenylation signal sequence have less than 70%, 65%, 60%, 55%, 50%, 45% or 40% sequence identity,
wherein the first and second recombinant polyadenylation signal sequence each have a sequence length of less than 100 nucleotides.

6. The recombinant nucleic acid of claim 5, further comprising:
(c) a third recombinant transcriptional unit comprising a third nucleotide sequence encoding a third polypeptide operably linked to a third recombinant polyadenylation signal sequence,
wherein the first and second recombinant polyadenylation signal sequence individually have less than 70%, 65%, 60%, 55%, 50%, 45% or 40% sequence identity with the third recombinant polyadenylation signal sequence,
wherein the third recombinant polyadenylation signal sequence has a sequence length of less than 100 nucleotides.

7. The recombinant nucleic acid of claim 5 or 6, wherein the first, second, and where present third recombinant polyadenylation signal sequence cannot engage in DNA strand exchange to form a recombination intermediate.

8. The recombinant nucleic acid of any one of claims 5-7, wherein the first recombinant transcriptional unit is a recombinant transcriptional unit according to any one of claims 1-4, and wherein the second recombinant transcriptional unit is a recombinant transcriptional unit according to any one of claims 1-4, and wherein where present the third recombinant transcriptional unit is a recombinant transcriptional unit according to any one of claims 1-4.

9. The recombinant nucleic acid of any one of claims 5-8, wherein (a) the first recombinant transcriptional unit further comprises a first promoter operably linked to the nucleotide sequence encoding the first polypeptide, and (b) the second recombinant transcriptional unit further comprises a second promoter operably linked to the nucleotide sequence encoding the second polypeptide,
wherein the first and second promoter have less than 70%, 65%, 60%, 55%, 50%, 45% or 40% sequence identity.

10. The recombinant nucleic acid of any one of claims 6-9, wherein:
(c) where present the first recombinant transcriptional unit further comprises a first promoter operably linked to the nucleotide sequence encoding the first polypeptide,
wherein the first and second promoter have less than 80%, 79%, 78%, 77%, 76%, 75%, 74%, 73%, 72%, 71%,70%, 65%, or 60% sequence identity with the third promoter.

11. The recombinant nucleic acid of any one of claims 5-10, wherein the recombinant nucleic acid comprises a first vector comprising the first recombinant transcriptional unit, and a second vector comprising the second recombinant transcriptional unit, and where a third recombinant transcriptional unit is present a third vector comprising the third recombinant transcriptional unit.

12. A host cell comprising the recombinant transcriptional unit of any one of claims 1-4 and/or the recombinant nucleic acid of any one of claims 5-11.

13. A recombinant viral vector comprising a vector genome, wherein the vector genome comprises in 5′ to 3′ order:
(i) a 5′ ITR sequence,
(ii) a promoter sequence,
(iii) a sequence encoding a polypeptide,
(iv) a recombinant polyadenylation signal sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, and
(v) a 3′ ITR sequence.

14. The recombinant viral vector of claim 13, wherein the recombinant polyadenylation signal sequence is selected from the group consisting of SEQ ID NO:6, SEQ ID NO:9, and SEQ ID NO:12.

15. The recombinant viral vector of claim 13 or 14, wherein the recombinant viral vector is selected from the group consisting of a retroviral vector, an adenoviral vector, a helper-dependent adenoviral vector, a hybrid adenoviral vector, a herpes simplex virus vector, a lentiviral vector, a poxvirus vector, an Epstein-Barr virus vector, a vaccinia virus vector, a human cytomegalovirus vectors, a lentiviral vector, an adenoviral vector or an adeno-associated virus (AAV) vector, or a recombinant variant derived therefrom.

16. The recombinant viral vector of any one of claims 13-15, wherein the recombinant viral vector is a recombinant adeno-associated virus (rAAV) vector.

17. A method of producing a polypeptide of interest, the method comprising the steps of
(a) providing the host cell of claim 12,
(b) incubating the host cell under conditions suitable for expression of the polypeptide,
(c) recovering the polypeptide of interest from the cell culture.

18. A method of producing a polypeptide of interest, the method comprising the steps of
(a) providing a host cell comprising the recombinant nucleic acid of any one of claims 5-11, wherein the polypeptide of interest is the first polypeptide, and wherein the second and where present third polypeptide are required for or improve the production of the polypeptide of interest,
(b) incubating the host cell under conditions suitable for expression of the first, second, and where present third polypeptide,
(c) recovering the polypeptide of interest from the cell culture, and optionally
(d) formulating the recovered polypeptide of interest for therapeutic use.

19. The method of claim 17 or 18, wherein the host cell is selected from the group consisting of a CHO cell, a BHK cell, a HEK cell, and a Sp2/0 cell.


SEQUENCE LISTING (19 sequences)

SEQ ID NO: 1 - DNA, 69 bp, synthetic construct
  source 1..69: mol_type = other DNA
tgagcatctgacttctggctaataaaatatctttatttagcatacatctgtgtgttggttttttgtgtg

SEQ ID NO: 2 - DNA, 95 bp, synthetic construct
  source 1..95: mol_type = other DNA
tattgactgctctgagaaagttgatttgagcatctgacttctggctaataaaatatctttatttagcatacatctgtgtgttggttttttgtgtg

SEQ ID NO: 3 - DNA, 95 bp, synthetic construct
  source 1..95: mol_type = other DNA
tattgactgctctgagaaagttgatttgagcatctgacttctggctaataaaatatctttatttagtcaacatctgtgtgttggttttttgtgtg

SEQ ID NO: 4 - DNA, 69 bp, synthetic construct
  source 1..69: mol_type = other DNA
tgaacatctgatggtctttgaataaagtctgagtgagtggcatacatctgtgtgttggttttttgtgtg

SEQ ID NO: 5 - DNA, 95 bp, synthetic construct
  source 1..95: mol_type = other DNA
acagacagtattgcttacgagttgattgaacatctgatggtctttgaataaagtctgagtgagtggcatacatctgtgtgttggttttttgtgtg

SEQ ID NO: 6 - DNA, 95 bp, synthetic construct
  source 1..95: mol_type = other DNA
acagacagtattgcttacgagttgattgaacatctgatggtctttgaataaagtctgagtgagtggtcaacatctgtgtgttggttttttgtgtg

SEQ ID NO: 7 - DNA, 69 bp, synthetic construct
  source 1..69: mol_type = other DNA
tgagcatctcctttaatcataataaaatctgtgatttctagcaacatctgtgtgttggttttttgtgtg

SEQ ID NO: 8 - DNA, 95 bp, synthetic construct
  source 1..95: mol_type = other DNA
atccagtcgtgtagttcttattaccttgagcatctcctttaatcataataaaatctgtgatttctagcaacatctgtgtgttggttttttgtgtg

SEQ ID NO: 9 - DNA, 95 bp, synthetic construct
  source 1..95: mol_type = other DNA
atccagtcgtgtagttcttattaccttgagcatctcctttaatcataataaaatctgtgatttctacagacatctgtgtgttggttttttgtgtg

SEQ ID NO: 10 - DNA, 69 bp, synthetic construct
  source 1..69: mol_type = other DNA
tgaacatctgatagtaaattaataaaaactgtgtgaagtgcatacatctgtgtgttggttttttgtgtg

SEQ ID NO: 11 - DNA, 95 bp, synthetic construct
  source 1..95: mol_type = other DNA
atcacggcactacactcgttgctttatgaacatctgatagtaaattaataaaaactgtgtgaagtgcatacatctgtgtgttggttttttgtgtg

SEQ ID NO: 12 - DNA, 95 bp, synthetic construct
  source 1..95: mol_type = other DNA
atcacggcactacactcgttgctttatgaacatctgatagtaaattaataaaaactgtgtgaagtgtcaacatctgtgtgttggttttttgtgtg

SEQ ID NO: 13 - DNA, 49 bp, synthetic construct
  source 1..49: mol_type = other DNA
aataaaatatctttattttcattacatctgtgtgttggttttttgtgtg

SEQ ID NO: 14 - DNA, 34 bp, synthetic construct
  source 1..34: mol_type = other DNA
aaataaaatacgaaatgaaataaaatacgaaatg

SEQ ID NO: 15 - DNA, 122 bp, synthetic construct
  source 1..122: mol_type = other DNA
tacttgtttattgcagcttataatggttacaaataaagcaatagcatcacaaatttcacaaataaagcatttttttcactgcattctagttgtggtttgtccaaactcatcaatgtatctta

SEQ ID NO: 16 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 17 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 18 - skipped

SEQ ID NO: 19 - DNA, 513 bp, synthetic construct
  source 1..513: mol_type = other DNA
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg